(19) United States
(12) Patent Application Publication
(10) Pub. No.: US 2012/0003263 A1
(43) Pub. Date: Jan. 5, 2012

(54) RECOMBINANT RACCOON POX VIRUS VACCINE AGAINST HIGHLY PATHOGENIC AVIAN INFLUENZA

(75) Inventors: Jorge E. Osorio, Mount Horeb, WI (US); Keith P. Iams, Frederick, MD (US); Brock Adam Bakke, Madison, WI (US); Shi-Hsia Hwa, Madison, WI (US)

(73) Assignee: WARF - Wisconsin Alumni Research Foundation, Madison, WI (US)

(21) Appl. No.: 13/132,862

(22) PCT Filed: Dec. 4, 2009
(86) PCT No.: PCT/US09/06399

§ 371 Date: Sep. 22, 2011

Related U.S. Application Data
(60) Provisional application No. 61/120,246, filed on Dec. 5, 2008.

(51) Int. Cl. A61K 39/145; A61P 31/16; A61P 37/04; A61K 39/295

(52) U.S. Cl. 424/199.1

(57) ABSTRACT

Vaccines comprising one or more recombinant raccoon pox viruses and methods of using those vaccines to immunize avians.

### CROSS-REFERENCE TO RELATED APPLICATIONS

This application claims the benefit of the filing date of U.S. application Ser. No. 61/120,246, filed on Dec. 5, 2008, the disclosure of which is incorporated by reference herein.

### BACKGROUND

The most deadly pandemic in modern history, killing up to 50 million worldwide, was caused by the 1918 “Spanish flu”, a virus which likely emerged from a direct avian to human transmission (DHHS, 2006; Taubenberger et al., 2005). More recently, H5N1 avian influenza was directly transmitted from birds to humans in Hong Kong in 1997 causing the death of six of the 18 people infected. In December 2003, this virus began to spread in poultry rapidly throughout Southeast Asia, and has since spread to Africa, the Middle East, and Europe, infecting 387 people to date, and causing massive economic losses in the poultry industry (DHHS, 2006; de Jong et al., 1997). One step in preventing a pandemic threat and controlling the spread of H5N1 influenza virus is an effective vaccine for domesticated animals that can be used to vaccinate fowl.

Influenza A vaccines tested in experimental studies and field usage in domesticated animals, specifically fowl, can be organized into four groups; inactivated whole influenza viruses, in vitro expressed antigen, nucleic acid vaccines, and in vivo expressed antigen (Swayne, 2008). Inactivated virus for highly pathogenic avian influenza is difficult to grow and even virus created by reverse genetics and reassortment still does not address the general problem of an inactivated vaccine failing to activate the cellular arm of the immune system and the high labor cost for parenteral administration. It is also difficult to differentiate infected from vaccinated animals (DIVA), and this is an integral part of avian influenza oversight. In vitro expressed antigen is very dependent on the quantity of each dose and the adjuvant system. Moreover, the cost of delivery and the need to identify better promoters in avian hosts has lent impetus to the use of nucleic acid vaccines.

Vaccines that provide protection soon after a single dose and do not require a subsequent booster vaccination are preferable for emergency administration in epizootics. In addition, preventing infection and blocking shedding of influenza virus in vaccinates is important for controlling the spread of avian influenza to non-vaccinated birds or humans. Further, a vaccine that does not interfere with DIVA serosurveillance is also desirable (Suarez et al., 2006; Tumpey et al., 2005; Veits et al., 2008: Capua et al., 2007), to avoid culling large numbers of animals whose infection status is unknown. An advantage of recombinant viral vectors expressing only the influenza HA protein is that they enable DIVA surveillance using test kits which detect antibodies to other avian influenza proteins, e.g., anti-nucleoprotein (NP) or matrix (M) antibodies (Swayne et al., 2007). However, recombinant vaccines based on avian pathogens such as fowlpox virus or Newcastle Disease virus may have significantly lower efficacy in chickens with pre-existing immunity to the vector (Swayne et al., 2000).

### SUMMARY OF THE INVENTION

An effective vaccine against highly pathogenic avian influenza (HPAI) which can be rapidly administered in the case of an outbreak is needed to protect both livestock and human health. As described hereinbelow, a recombinant raccoon pox virus (RCN) expressing the hemagglutinin (HA) gene of the Clade 1 influenza virus A/Vietnam/1203/04 was prepared. RCN-HA administered by the intramuscular and intradermal routes protected mice against lethal challenge with Vietnam/1203. To determine the suitability of RCN for use in avian species, the replication of RCN in chicken embryo fibroblasts (CEF) and chickens was studied using highly pathogenic avian influenza virus H5N1 hemagglutin (HA) as a model antigen and luciferase (luc) as a reporter gene. While RCN replicated to low levels in CEFs, it efficiently expressed recombinant proteins. Biophotonic in vivo imaging of one-week-old chicks with RCN-luc showed strong expression of the luc reporter gene lasting up to 3 days post-infection. In addition, groups of two-week-old chicks received RCN-HA by intramuscular, intradermal, oral, and oculonasal routes. No illness or gross lesions (other than a vaccination “take” in the wing-web vaccinated chicks) were observed, and chick growth was not impaired compared to controls, indicating that RCN is safe in juvenile chickens. Moreover, chicks vaccinated with RCN-HA by the wing-web and intramuscular routes developed strong anti-raccoon pox titers (1:640-1:10240), and the anti-HA IgY antibody responses were comparable with that for inactivated influenza virus. The immunogenicity of RCN-HA in mammals and birds, and by multiple routes of administration, is advantageous for mass vaccination in the case of an HPAI outbreak.

The invention thus provides a composition comprising a live recombinant raccoon pox virus, the genome of which comprises at least one expression cassette having a promoter operably linked to a heterologous open reading frame comprising a nucleotide sequence for an antigen of a pathogen, e.g., an avian viral, bacterial or fungal pathogen, that elicits neutralizing antibodies and/or a cytotoxic T cell response. In one embodiment, the composition includes more than one live recombinant raccoon pox virus, e.g., different isolates having different antigens or different isolates, at least one of which encodes an adjuvant. In one embodiment, the composition includes at least one live recombinant raccoon pox virus and an isolated pathogen, e.g., inactivated pathogen, isolated antigenic protein of a pathogen or isolated nucleic acid encoding an antigenic protein of a pathogen. Once the recombinant virus infects cells of a host animal, the antigen is expressed in an amount effective to induce an immune response to a pathogen having that antigen. In one embodiment, the antigen is highly variant, such as the hemagglutinin (HA) of influenza virus. In one embodiment, the pathogen is a pathogen of birds (avians). In one embodiment, the pathogen is highly pathogenic, e.g., infection with the pathogen is associated with at least 80% or greater, e.g., 85%, 90% or greater, mortality. In one embodiment, the live recombinant raccoon pox virus may be obtained from a culture of isolated mammalian cells transfected or transformed with a recombinant raccoon pox virus genome comprising the at least one expression cassette. Any cell, e.g., any avian or mammalian cell, such as a human, canine, bovine, equine, feline, swine, ovine, mink, or non-human primate cell, including mutant cells, which supports efficient replication of raccoon pox virus can be employed to isolate and/or propagate the viruses. In another embodiment, host cells are continuous mammalian or avian cell lines or cell strains.

A composition of the invention, such as a vaccine, e.g., for in ovo, mucosal, dermal or intramuscular administration, may include from 1×10⁵ to 1×10⁹ plaque forming units (PFU) or TCID₅₀, e.g., from 1×10⁶ to 1×10⁹ PFU or TCID₅₀, which may be administered as a single dose or in two or more doses, or each dose may include from 1×10⁵ to 1×10⁹ PFU or TCID₅₀, e.g., from 1×10⁶ to 1×10⁸ PFU or TCID₅₀. For instance, each dose may have the same number of PFU or TCID₅₀, or the booster dose(s) may have higher or lower amounts relative to the initial (priming) dose. The priming dose and/or booster dose(s) may include an adjuvant. In one embodiment, the vaccine confers from 70 to 100% protection against homologous challenge. In one embodiment, the protection provided by a vaccine of the invention is substantially similar to protection provided by an inactivated preparation of a corresponding pathogen. In one embodiment, the vaccine confers from 30 to 100% protection against heterologous challenge, e.g., challenge with a different strain of the pathogen (cross protection).

In one embodiment, a vaccine of the invention encodes an influenza virus HA and/or NA, which may induce a humoral response, a cellular response, or both, and encodes an influenza virus NP, which due to its relative conservation, likely provides cross-protection.

In one embodiment, the promoter is a heterologous promoter, for example, a non-raccoon pox virus promoter, for instance, one selected to provide constitutive expression. In one embodiment, the promoter is a raccoon pox virus promoter, e.g., one that is part of the expression cassette. In one embodiment, the heterologous open reading frame encodes a fusion protein, such as one having a heterologous secretory signal at the N-terminus of the antigen. In another embodiment, the heterologous open reading frame encodes an antigen having a native (homologous) secretory signal. In one embodiment, the heterologous open reading frame encodes a fusion protein, including one having a heterologous membrane anchoring peptide. In another embodiment, the heterologous open reading frame encodes an antigen having a native membrane anchoring peptide.

Further provided is a method to immunize an animal against a pathogen, e.g., an avian viral, bacterial or fungal pathogen. The method includes administering to an animal, e.g., a bird, human, canine, feline, bovine, equine, swine, or ovine, an effective amount of one or more live recombinant raccoon pox viruses, the genome of at least one of which comprises at least one expression cassette having a promoter operably linked to a heterologous open reading frame comprising a nucleotide sequence for an antigen of a pathogen that elicits neutralizing antibodies and/or a cellular immune response. In one embodiment, the antigen is highly variant, such as the HA of influenza virus. In one embodiment, the pathogen is a pathogen of birds. In one embodiment, the pathogen is highly pathogenic, e.g., infection with the pathogen is associated with at least 80% or greater, e.g., 85%, 90% or greater, mortality. In one embodiment, the animal is not a mammal, e.g., the animal is an avian. For example, the effective amount may be from 1×10⁵ to 1×10⁹ PFU or TCID₅₀, e.g., from 1×10⁶ to 1×10⁸ PFU or TCID₅₀, which may be administered as a single dose or in two or more doses, or each dose may include from 1×10⁵ to 1×10⁹ PFU or TCID₅₀, e.g., from 1×10⁶ to 1×10⁸ PFU or TCID₅₀. For instance, each dose may have the same number of PFU or TCID₅₀, or the booster dose(s) may have higher or lower amounts relative to the initial (priming) dose. The priming dose and/or booster dose(s) may include an adjuvant. In one embodiment, the method includes administering to the mucosa of an avian, e.g., orally administering, an effective amount of one or more live recombinant raccoon pox viruses, the genome of at least one of which comprises at least one expression cassette having a promoter operably linked to a heterologous open reading frame comprising a nucleotide sequence for an antigen of a pathogen that elicits neutralizing antibodies and/or a cellular immune response. For example, the effective amount may be from 1×10⁵ to 1×10⁸ PFU or TCID₅₀, e.g., from 1×10⁶ to 1×10⁷ PFU or TCID₅₀, which may be administered as a single dose or in two or more doses, or each dose may include from 1×10⁵ to 1×10⁸ PFU or TCID₅₀, e.g., from 1×10⁶ to 1×10⁷ PFU or TCID₅₀. For instance, each dose may have the same number of PFU, or the booster dose(s) may have higher or lower amounts relative to the initial dose. The initial booster may be administered from 2 to 8 weeks after the priming dose, for instance 3 to 4 weeks after the priming dose. The priming dose and/or booster dose(s) may include an adjuvant.

In one embodiment, the method includes administering to an egg of an avian an effective amount of one or more live recombinant raccoon pox viruses, the genome of at least one of which comprises at least one expression cassette having a promoter operably linked to a heterologous open reading frame comprising a nucleotide sequence for an antigen of a pathogen that elicits neutralizing antibodies and/or a cellular immune response. For example, the effective amount may be from 1×10⁵ to 1×10⁹ PFU or TCID₅₀, e.g., from 1×10⁶ to 1×10⁸ PFU or TCID₅₀, which may be administered as a single dose or in two or more doses, or each dose may include from 1×10⁵ to 1×10⁹ PFU or TCID₅₀, e.g., from 1×10⁶ to 1×10⁸ PFU or TCID₅₀. For instance, each dose may have the same number of PFU or TCID₅₀, or the booster dose(s) may have higher or lower amounts relative to the initial (priming) dose. The priming dose and/or booster dose(s) may include an adjuvant. In one embodiment, the method includes administering to the mucosa of an avian, e.g., orally administering, an effective amount of one or more live recombinant raccoon pox viruses, the genome of at least one of which comprises at least one expression cassette having a promoter operably linked to a heterologous open reading frame comprising a nucleotide sequence for an antigen of a pathogen that elicits neutralizing antibodies and/or a cellular immune response. For example, the effective amount may be from 1×10⁵ to 1×10⁸ PFU or TCID₅₀, e.g., from 1×10⁶ to 1×10⁷ PFU or TCID₅₀, which may be administered as a single dose or in two or more doses, or each dose may include from 1×10⁵ to 1×10⁸ PFU or TCID₅₀, e.g., from 1×10⁶ to 1×10⁷ PFU or TCID₅₀. For instance, each dose may have the same number of PFU, or the booster dose(s) may have higher or lower amounts relative to the initial dose. The initial booster may be administered from 2 to 8 weeks after the priming dose, for instance 3 to 4 weeks after the priming dose. The priming dose and/or booster dose(s) may include an adjuvant. In one embodiment, mucosal delivery of the recombinant virus and adjuvant is employed, e.g., a recombinant virus encoding influenza HA and an adjuvant that is a modified toxin, which adjuvant may be delivered via a live recombinant raccoon pox virus.

Also provided is a method to inhibit or treat pathogen infection, e.g., an avian viral, bacterial or fungal pathogen of an animal. The method includes administering to an animal an effective amount of a live recombinant raccoon pox virus, the genome of which comprises at least one expression cassette having a promoter operably linked to a heterologous open reading frame comprising a nucleotide sequence for an antigen of a pathogen that elicits neutralizing antibodies and/or a cellular immune response. In one embodiment, the animal is not a mammal, e.g., an avian.

Also provided is a method to immunize an avian or an egg thereof against a pathogen. The method includes administering to the avian or an egg thereof an effective amount of a live recombinant raccoon pox virus, the genome of which comprises at least one expression cassette having a promoter operably linked to a heterologous open reading frame comprising a nucleotide sequence for an antigen of a pathogen that elicits neutralizing antibodies and/or a cellular immune response. The immunized avian may be one of a population of avians, e.g., a flock of chickens, where at least one of the population has symptoms of pathogen infection or anti-pathogen antibodies. In one embodiment, the antigen is highly variant, such as the HA of influenza virus. In one embodiment, the pathogen is a pathogen of birds. In one embodiment, the pathogen is highly pathogenic, e.g., infection with the pathogen is associated with at least 80% or greater, e.g., 85%, 90% or greater, mortality. In one embodiment, eggs of a chicken, e.g., 10 day old eggs, are immunized.

Further provided is a diagnostic method. The method includes providing a physiological sample having antibodies of an animal suspected of being exposed to a pathogen such as influenza virus and immunized with a live recombinant raccoon pox virus, the genome of which comprises at least one expression cassette having a promoter operably linked to a heterologous open reading frame comprising a nucleotide sequence for an antigen of the pathogen that induces the production of neutralizing antibodies and/or a cellular immune response. In one embodiment, the antigen is an influenza virus antigen. In one embodiment, the physiological sample may be a physiological fluid sample such as a serum, bile, or sputum sample from an animal suspected of being or having been infected, e.g., an avian. In one embodiment, the sample is a blood or nasal secretion sample. In one embodiment, the method includes determining the presence or amount of antibodies to the antigen of the pathogen encoded by the recombinant virus. In one embodiment, it is determined whether the animal was exposed to the pathogen by determining the presence or amount of antibodies to the pathogen other than that encoded by the recombinant virus. In one embodiment, the antibodies are IgY, IgG or IgA antibodies. In one embodiment, the method includes determining the presence or amount of anti-raccoon pox virus antibodies.

The invention also provides a method to induce an immune response to an antigen. The method includes administering to a nonmammalian animal an effective amount of a live recombinant raccoon pox virus, the genome of which comprises at least one expression cassette having a promoter operably linked to a heterologous open reading frame comprising a nucleotide sequence for the antigen. In one embodiment, the method includes administering to a nonmammalian animal an amount of a live recombinant raccoon pox virus, the genome of which comprises at least one expression cassette having a promoter operably linked to a heterologous open reading frame comprising a nucleotide sequence for an antigen of the pathogen that induces an immune response to the pathogen, effective to immunize an animal against pathogen infection or replication.

### BRIEF DESCRIPTION OF THE FIGURES

FIG. 1. Schematic of insertion of P11-IRES-tPA-HA expression cassette into racconpox virus (RCNV) thymidine kinase (TK) gene by homologous recombination.

FIG. 2. Western blot analysis of HA expression in infected Vero cells. Lanes: 1) 25 μL RCN-HA infected Vero cell lysate. 2) 2.5×10⁷ PFU purified RCN-HA vaccine. 3) 25 μL RCNΔTK infected Vero cell lysate. 4) 0.2 μg recombinant H5-HA protein (BEI Resources).

FIG. 3. Safety of raccoon pox viral vector in SCID mice. All the groups of mice were infected with 1×10⁸ PFU of virus except the group that was infected with vaccinia-Wyeth, which received 1×10⁶ PFU of the virus. Mice were weighed individually and averages plotted. Mice died naturally or were euthanized after 20% weight loss or a body conditioning score less than two (BCS<2).

FIG. 4. ELISA for anti-HA IgG titers. Plates were coated with recombinant purified HA protein cloned from A/Vn/1203/04 and murine blood samples were added.

FIG. 5. Kaplan-Meier survival analysis of mice immunized with RCN/HA constructs after intranasal (IN) and intramuscular (IM) challenge with 10 LD₅₀ of A/Vn/1203/04. Differences in survival rates between challenged groups were analyzed by Kaplan-Meier method with the log-rank (Mantel-Cox) test. Differences with P values of 0.05 or less were considered significant.

FIG. 6. Kaplan-Meier survival analysis of mice immunized with RCN/HA constructs after intranasal challenge with 10 LD₅₀ of A/Vn/1203/04. Immunized mice were challenged via the intransal route by inoculating mice with 1×10⁴ (10 LD₅₀). Differences in survival rates between challenged groups were analyzed by Kaplan-Meier method with the log-rank (Mantel-Cox) test. Differences with P values of 0.05 or less were considered significant.

FIG. 7. Replication of RCN-HA in Vero and primary CEF cells. Replication was calculated as the total amount of virus in a sample (viral titer multiplied by sample volume) divided by the amount of virus in the original inoculum. Results represent the mean of two independent experiments.

FIG. 8. Expression of HA protein in CEF cells and culture supernatant. CC, uninfected cell controls harvested at 120 hours; C, cell pellet (150 μL from 2 mL total sample volume); S, supernatant (150 μL from 8 mL total sample volume).

FIG. 9. Luciferase activity in embryo tissue and allantoic fluid from eggs infected with RCN-luc at 24 h.p.i. Individual 10 μL sample volumes were measured in duplicate and averaged. Bars show means±standard deviation. A) Eggs harvested at 24 hours post-infection. B) Eggs harvested at 48 hours post-infection.

FIG. 10. RCN-luc titers in embryo tissue and allantoic fluid from infected eggs. Samples were titrated on Vero cells. Bars show means±standard deviation. A) Eggs harvested at 24 hours post-infection. B) Eggs harvested at 48 hours post-infection.

FIG. 11. Biophotonic imaging of chicks inoculated with RCN-luc by A) IM injection and B) ID injection at 1, 3, and 5 days post-inoculation. The left-most chick in each image set is an RCN-HA vector control. Red circles delineate regions of interest (ROIs) used to calculate radiance. False-color scales showing total photon flux vary between sets of 4 birds but are set to the same minima and maxima for each set.

FIG. 12. Radiance (normalized photon emission in units of photons/s/cm²/steradian) in ROIs of luminescent chick images from days 1, 3, 5, and 7 post-inoculation. Open squares ═RCN-HA IM; solid circles ═RCN-luc IM; open triangles ═RCN-luc ID.

FIG. 13. Anti-HA titers in chick serum four weeks after initial vaccination and two weeks after booster vaccination. The capture antigen is purified recombinant H5 HA protein. Bars show means±standard deviation.

FIG. 14. Anti-RCN titers in chick serum one week after initial vaccination and two weeks after booster vaccination. The capture antigen is whole inactivated RCN-wt. Bars show means±standard deviation.

### DETAILED DESCRIPTION OF THE INVENTION

### Definitions

As used herein, the term “isolated” refers to in vitro preparation and/or isolation of a nucleic acid molecule, e.g., vector or plasmid, peptide or polypeptide (protein), or virus of the invention, so that it is not associated with in vivo substances, or is substantially purified from in vitro substances. An isolated virus preparation is generally obtained by in vitro culture and propagation, and is substantially free from other infectious agents.

As used herein, “substantially purified” means the object species is the predominant species, e.g., on a molar basis it is more abundant than any other individual species in a composition, and in one embodiment is at least about 80% of the species present, and optionally 90% or greater, e.g., 95%, 98%, 99% or more, of the species present in the composition.

As used herein, “substantially free” means below the level of detection for a particular infectious agent using standard detection methods for that agent.

A “recombinant” virus is one which has been manipulated in vitro, e.g., using recombinant DNA techniques, to introduce changes to the viral genome.

As used herein, the term “recombinant nucleic acid” or “recombinant DNA sequence or segment” refers to a nucleic acid, e.g., to DNA, that has been derived or isolated from a source, that may be subsequently chemically altered in vitro, so that its sequence is not naturally occurring, or corresponds to naturally occurring sequences that are not positioned as they would be positioned in the native genome. An example of DNA “derived” from a source, would be a DNA sequence that is identified as a useful fragment, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

An “avian” includes domestic, wild and game birds, such as chickens, turkeys and other gallinaceous birds; ducks, geese, swans and other waterfowl, ostriches and other ratites. Exotic avians include but are not limited to cockatiels, parakeets, macaws, conures, African grays, lovebirds, and other parrots (psittacines); canaries, finches and other passerines; hawks, eagles, falcons and other raptors.

As used herein, the term “heterologous” nucleic acid sequence or protein refers to a sequence that relative to a reference sequence has a different source, e.g., originates from a foreign species, or, if from the same species, it may be substantially modified from the original form.

### Methods and Viral Vectors

The invention encompasses a variety of types of viral vectors, each of which may include one or more heterologous nucleic acid sequences that encode an antigen from a pathogen and methods of using these vectors, alone or in combination with one another or other vectors or antigenic preparations, or combinations thereof, for example, to immunize animals against the pathogen(s) from which the antigen(s) were obtained (thereby reducing the animal's risk of becoming infected) or to treat animals who have already become infected. The immunization methods may elicit both cell-mediated and humoral immune responses that may substantially prevent the infection or limit its extent or impact on the animal's health. Immunization can result in protection against subsequent challenge by the pathogen; a human or other animal, such as a domesticated or farm animal, is immunized if they mount an immune response that protects them (partially or totally) from the manifestations of infection (i.e., disease) caused by a pathogen. Thus, an immunized animal will not be infected by the pathogen or will be infected to a lesser extent than one would expect in the absence of immunization. In one embodiment, an immunized animal will also not be infected by a related pathogen (cross-protection) or will also be infected to a lesser extent than one would expect in the absence of immunization.

Raccoon pox virus is an attractive vaccine vector due to the high levels of antigen expression, the unique ability to tolerate large inserts of up to 27 kilobases in length, and the low seroprevalence in domesticated animals. The use of raccoon pox virus as a vaccine vector allows for DIVA surveillance, and because it does not cross react with fowlpox virus, raccoon pox virus based vaccines will not be neutralized in animals exposed to fowlpox virus or vaccinated with fowlpox virus derived vaccines. Moreover, raccoon pox virus has been shown to be a safe and effective vaccine in mice, rabbits, piglets and cats (Osorio et al., 2003a; Osorio et al., 2003b), i.e., it has a wide host range, and as discussed below, it replicates in chickens. Recombinant raccoon pox viruses of the invention may encode any heterologous antigen of a pathogen, e.g., an antigen useful to induce an immune response to avian influenza in a population suspected of or at risk of exposure to avian influenza.

A recombinant raccoon pox virus of the invention may be derived from a naturally-occurring or designated wild-type virus strain that is a DNA cytoplasmic pox virus, e.g., one which does not integrate into a host cell genome. The antigen encoded by the recombinant raccoon pox virus can be from any pathogen including any virus, bacteria, parasite or fungi that generates a pathological condition in an animal. The virus can be, for example, a herpesvirus, an influenza virus, a orthomyxovirus, a rhinovirus, a picornavirus, an adenovirus, a paramyxovirus, a coronavirus, a rhabdovirus, a togavirus, a flavivirus, a bunyavirus, a rubella virus, a reovirus, a measles virus, a hepadna virus, a filovirus, or a retrovirus (including a human immunodeficiency virus; including all clades of HIV-1 and HIV-2 and modifications thereof). The bacteria can be, for example, a mycobacterium (e.g., M. tuberculosis, which causes tuberculosis or M. leprae, which causes leprosy), a spirochete, a rickettsia, a chlamydia, or a mycoplasma. The parasite can be, for example, a parasite that causes malaria, and the fungus can be, for example, a yeast or mold. Antigens for avian vaccines include antigens from the pathogens or associated with the diseases listed in Table 1. In one embodiment, the antigen is a glycoprotein. In one embodiment, the antigen is a viral capsid protein. In one embodiment, the antigen is a nonstructural protein, e.g., a protein that is not a viral polymerase. For example, the antigen may be any of the M, E or C proteins of West Nile virus, any of the N protein, P protein, M protein, F protein or glycoprotein of a paramyxovirus, or the capsid or a nonstructural protein of an astrovirus. In one embodiment, the antigen may be a toxin protein or a modified toxin protein, e.g., from Clostridium botulinum.

TABLE 1
Pathogen/Disease

West Nile virus
Astrovirus
Newcastle Disease (Paramyxovirus 1)
Ornithobacterium Infection, ORT
Laryngotracheitis
Fowl cholera
Avian encephalomyelitis
Infectious bronchitis
Marek's disease
Avian Rhinotracheitis ‘Swollen Head Syndrome’
Clostridium botulinum
Campylobacter Infection
Chlamydia psittaci
Eimeria/Coccidiosis
Equine Encephalitis (EEE, WEE, VEE)
Duck Viral Hepatitis
Cryptosporidia
Erysipelothrix insidiosa
Pasteurella multocida
Goose Parvovirus (Derzsy's Disease)
Viral Arthritis
Turkey Rhinotracheitis
Transmissible Enteritis
Streptococcus bovis
Staphylococcosis, Staphylococcal Arthritis, Bumble
Foot
Salmonellosis, S. Enteritidis and S. Typhimurium
infections
Salmonella Pullorum, Pullorum Disease, ‘Bacillary
White Diarrhoea’
Salmonella Gallinarum, Fowl Typhoid
Rotavirus
Pseudotuberculosis
Paramyxovirus-6
Paramyxovirus-3
Paramyxovirus 2 - Yucaipa Disease
Ornithobacterium Infection, ORT
Marek's disease (Herpes virus)
Mycoplasma

One of ordinary skill in the art will recognize that the methods described herein can be used to generate protective or therapeutic immune responses against many other pathogens. The antigen (or immunogen) may be a structural component of the pathogen; the antigen (or immunogen) may be glycosylated, myristoylated, or phosphorylated; the antigen (or immunogen) may be one that is expressed intracellularly, on the cell surface, or secreted (antigens that are not normally secreted may be linked to a signal sequence that directs secretion). In one embodiment, an antigen that elicits neutralizing antibodies when administered to an animal and another antigen that elicits a cellular response when administered to an animal, are employed in the recombinant raccoon pox viruses of the invention to generate protective or therapeutic immune responses. In one embodiment, the pathogen is highly pathogenic, e.g., infection with the pathogen is associated with at least 80% or greater, e.g., 85%, 90% or greater, mortality. In one embodiment, the pathogen is a pathogen of birds. In one embodiment, the antigen is highly variant, such as the hemagglutinin (HA) of influenza virus. Where the antigen is obtained from, or derived from, the virus that causes influenza, the antigen can be all, or an antigenic portion of, for example, neuraminidase, nucleoprotein, or hemagglutinin, or a combination thereof, any or all of which may be expressed as a fusion protein. Antigens directed against any pathogenic condition may contain a mutation, so long as they retain the ability to induce or enhance an immune response that confers a protective or therapeutic benefit on the immunized animal. In one embodiment, the influenza virus antigen is one that elicits neutralizing antibodies when administered to an animal, e.g., HA, NA or M. In another embodiment, the influenza virus antigen is one that elicits a cytotoxic T cell response when administered to an animal, e.g., NP. In one embodiment, an antigen that elicits neutralizing antibodies when administered to an animal and another antigen that elicits a cellular response when administered to an animal are employed in the recombinant raccoon pox viruses of the invention.

In one embodiment, vaccine efficacy may be enhanced by employing a recombinant raccoon poxvirus with two or more different antigens, e.g., one that elicits neutralizing antibodies and another that elicits a cytotoxic T cell response, or employing two or more different recombinant raccoon poxviruses, e.g., at least one encoding an antigen that elicits neutralizing antibodies and another encoding an antigen that elicits a cytotoxic T cell response. In one embodiment, a vaccine of the invention encodes an influenza virus HA and/or NA, which may induce a humoral response, a cellular response, or both, and encodes an influenza virus NP, which due to its relative conservation, likely provides cross-protection.

In one embodiment, the recombinant raccoon pox viruses are substantially avirulent in the animal administered the virus, that is, the recombinant virus does not cause disease in the animal. Thus, raccoon pox viruses for use in preparing the recombinant viruses of the invention have a host range that extends beyond that of the natural host. For example, a raccoon pox virus may be used as a virus vector when the host is a mammal, a primate such as a human, as well as an avian.

A DNA fragment encoding an immunogenic peptide or polypeptide of a pathogen can be inserted into the genome of a suitable raccoon poxvirus by conventional recombinant techniques to produce recombinant pox viruses. For example, a DNA donor vector including a DNA insert encoding an immunogenic peptide or polypeptide with flanking DNA sequences homologous to sequences in the genome of the virus is employed. Other techniques that may be used include the use of a unique restriction endonuclease site that is naturally present or artificially inserted in the parental viral vector (see e.g., Mackett et al., 1982; and U.S. Pat. No. 5,093,258).

In one embodiment, the DNA fragment is inserted by conventional methods into the DNA donor vector, such as those suitable for use in a eukaryotic cell, such as a rodent cell. The donor vector includes viral DNA which is homologous to a segment of raccoon pox virus DNA at the site to which insertion of the fragment is desired, such as the TK gene (Lutze-Wallace et al., 1995), or the raccoon pox hemagglutinin gene (Carallaro et al., 1992) or intergenic regions, e.g., at a site or gene at which an insertion does not substantially alter the replication of the resulting virus. DNA encoding the immunogenic fragment can be inserted into the DNA donor vector adjacent and operably linked to suitable control elements in the vector such as a promoter, an enhancer, a ribosome binding site (IRES), leader, e.g., secretory, sequences, and/or membrane anchoring sequences. For prokaryotic vector insertion, exemplary DNA donor vectors generally include an origin of replication, such as the E. coli origin of replication, and a marker such as an antibiotic resistance gene for selection and propagation in a suitable prokaryotic host such as E. coli. The resulting DNA donor vector is then propagated by growth within a suitable prokaryotic host cell, isolated and purified if desired. Selectable marker genes are known in the art and include, for example, genes encoding proteins that confer antibiotic resistance on a cell in which the marker is expressed (e.g., resistance to kanamycin or ampicillin). The selectable marker is so-named because it allows one to select cells by virtue of their survival under conditions that, absent the marker, would destroy them. The selectable marker can be, but need not be, excised from the plasmid before it is administered to a patient. Similarly, plasmid vectors can be administered in a circular form, after being linearized by digestion with a restriction endonuclease, or after some of the vector “backbone” has been altered or deleted.

The DNA donor vector including the immunogenic fragment to be inserted into a desired raccoon pox virus is generally transfected into a suitable cell culture, e.g., a mammalian or primate cell line, that is infected with raccoon pox virus. Recombination between homologous DNA in the DNA donor vector and the pox virus genome forms a recombinant pox virus modified by the presence of the immunogenic fragment. In one embodiment, the site of the insertion does not substantially affect the viability of the recombinant virus (nonessential regions or genes, see, e.g., U.S. Pat. No. 6,294,176, the disclosure of which related to nonessential regions is incorporated by reference herein). The skilled artisan can readily identify such regions in the virus by, for example, randomly testing segments of viral DNA for regions that allow recombinant formation without affecting virus viability of the recombinant. As mentioned above, one region that can readily be used and is present in many viruses is the thymidine kinase (TK) gene. Viral viability can be readily tested by, e.g., viral plaque assay or a DNA replication assay involving tagging newly synthesized DNA with a detectably-labeled nucleotide (e.g., ³H-thymidine). Typically, viral viability may be assessed by comparing the viability of the recombinant virus to that of a control virus (i.e., no inserted DNA).

In one embodiment, expression of the inserted gene by the recombinant pox virus is via the presence of a native promoter which, after homologous recombination, is operably linked to the desired gene, i.e., in the proper relationship to the inserted gene. In another embodiment, expression of the inserted gene by the recombinant pox virus is via the presence of a heterologous promoter operably linked to the desired gene, i.e., in the proper relationship to the inserted gene. Heterologous promoters are well known in the art and can readily be selected depending on the host and the cell type you wish to target. Promoters known as strong promoters can be used. One such promoter is the cytomegalovirus (CMV) intermediate early promoter, although other (including weaker) promoters may be used without departing from the scope the present invention. For example, early or late promoters in the viral replication cycle may be employed. Artificial (synthetic or chimeric) pox promoter constructs containing appropriate promoter sequences may also be used. Enhancer elements can also be used in combination to increase the level of expression. Further, the use of inducible promoters, which are also well known in the art, is also envisioned. Thus, in one embodiment, the promoter may be modulated by addition of an external agent or subjecting the infected cells to a particular condition, and in turn to control the level of polypeptide being produced by the vectors. Similarly, strong polyadenylation signals may be selected for use in the DNA donor vector.

The vectors can further include a leader (secretory signal) sequence (e.g., a leader sequence that is a synthetic homolog of the tissue plasminogen activator gene leader sequence (tPA) or a native raccoon pox virus secretory signal) may be employed in the expression cassette). It will be appreciated that expression also may be enhanced by other methods known in the art including, but not limited to, optimizing the codon usage and multi-cistronic vectors may be used to express more than one immunogen.

Expression of the immunogenic fragment can be readily determined by several methods, including assaying samples of a suitable target cell or tissue by SDS-PAGE gel electrophoresis followed by Coomassie blue or silver staining; Western blot or other suitable immunological technique such as ELISA.

The recombinant viruses may be administered by scarification, as was conventionally done for small pox vaccination, or by other routes appropriate to the recombinant virus used. These may include among others, intramuscular, intradermal, mucosal, subcutaneous, and intravenous routes. Vaccination of a host organism with live recombinant virus is followed by replication of the virus within the host.

A specific immune response may be generated by administering between about 10⁵ to 10¹² PFU of the recombinant virus. In one embodiment, the host is an avian, e.g., a chicken, turkey or exotic bird. At least one interval thereafter, the immune response may be boosted by administering additional antigen to the host. In one embodiment, there is at least a second “boost.” The boosting antigen may be administered using the same pox virus vector, or as a whole protein, an immunogenic peptide fraction of the protein, another recombinant viral vector, or DNA encoding the protein or peptide. In one embodiment, the initial immunization and/or boosts include an adjuvant. Adjuvants include, for example, RIBI Detox (Ribi Immunochemical), QS21 (Aquila), incomplete Freund's adjuvant or many others including a genetic adjuvant such as Interleukin-15, or heat lable toxin subunit A/B. In one embodiment, the adjuvant is a protein or fragment thereof, e.g., an enterotoxin. In one embodiment, the adjuvant protein or fragment is encoded by a live recombinant raccoon pox virus, e.g., the same virus having the antigen of interest or a different virus. In another embodiment, the initial immunogen is a whole protein, an immunogenic peptide fraction of the protein, a non-raccoonpox virus vector or DNA encoding the protein or peptide, and the boosting agent is a recombinant raccoon pox virus. In one embodiment, a recombinant pox virus may encode two different heterologous antigens. In another embodiment, co-expression of two or more antigens by multiple recombinant vectors may be desirable.

In one embodiment, after initial administration(s) of the viral vector by one pox, a different pox virus, e.g., from a different pox family may be used for the following administrations (i.e., boosts). For example, initial administration(s) by raccoon pox may be followed by boosts from an avipox or vaccina, respectively, or by a suipox, or a different virus. Exemplary of such vectors are DNA or RNA viruses such as retroviruses, adenoviruses, herpes viruses or DNA-based vectors.

Further uses of the recombinant raccoon pox viruses described herein include the preparation of virus-like particles (VLPs), e.g., the expression of a subset of influenza virus genes may result in influenza VLPs that are highly immunogenic and nonreplicating, which may be useful in a vaccine.

The compositions described herein can be administered in a variety of ways including through any parenteral or topical route. For example, an individual can be inoculated by intravenous, intraperitoneal, intradermal, subcutaneous or intramuscular methods. Inoculation can be, for example, with a hypodermic needle, a nebulizer or needleless delivery devices such as those that propel a stream of liquid into the target site. The vaccine can be administered to a mucosal surface by a variety of methods including intranasal administration, e.g., nose drops or inhalants, or intrarectal or intravaginal administration by solutions, gels, foams, or suppositories. Alternatively, the vector comprising the vaccine insert can be orally administered in the form of a tablet, capsule, chewable tablet, food, e.g., food pellets or bait, water, syrup, emulsion, or the like. In an alternate embodiment, vectors can be administered transdermally, by passive skin patches, iontophoretic means, and the like.

Any physiologically acceptable medium can be used to introduce a vaccine into an animal. For example, suitable pharmaceutically acceptable carriers known in the art include, but are not limited to, sterile water, saline, glucose, dextrose, or buffered solutions. The media may include auxiliary agents such as diluents, stabilizers (i.e., sugars (glucose and dextrose were noted previously) and amino acids), preservatives, wetting agents, emulsifying agents, pH buffering agents, additives that enhance viscosity or syringability, colors, and the like. In one embodiment, the medium or carrier will not produce adverse effects, or will only produce adverse effects that are far outweighed by the benefit conveyed.

### Pharmaceutical Compositions

Pharmaceutical compositions of the present invention, suitable for inoculation, e.g., nasal, ocular, parenteral or oral administration, comprise one or more raccoon pox virus isolates, e.g., one or more live raccoon pox viruses or at least one live raccoon pox virus and one or more other pathogens, e.g., attenuated or inactivated viruses, a subunit thereof, isolated protein(s) thereof, and/or isolated nucleic acid encoding one or more proteins thereof, optionally further comprising sterile aqueous or non-aqueous solutions, suspensions, and emulsions. The compositions can further comprise auxiliary agents or excipients, as known in the art. The composition of the invention is generally presented in the form of individual doses (unit doses).

For example, for influenza virus vaccines, conventional vaccines generally contain about 0.1 to 200 μg, e.g., 30 to 100 μg, of influenza virus HA from each of the strains entering into their composition. The vaccine forming the main constituent of the vaccine composition of the invention may comprise a single raccoon pox virus encoding an influenza virus antigen, or one or more raccoon pox viruses encoding antigens from a combination of influenza viruses or combination of antigens, for example, at least two or three different influenza virus antigens.

Preparations for parenteral administration include sterile aqueous or non-aqueous solutions, suspensions, and/or emulsions, which may contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents.

When a composition of the present invention is used for administration to an individual animal, it can further comprise salts, buffers, adjuvants, or other substances which are desirable for improving the efficacy of the composition. For vaccines, adjuvants, substances which can augment a specific immune response, can be used. Normally, the adjuvant and the composition are mixed prior to presentation to the immune system, or presented separately, but into the same site of the organism being immunized. Materials suitable for use in vaccine compositions are known to the art.

In one embodiment, a composition of the invention is formulated with gelatin or other edible solid, semi-solid or aqueous components, or encapsulated in edible components, e.g., formulated with or encapsulated in food-flavored bait for field immunization of wildlife.

A pharmaceutical composition according to the present invention may further or additionally comprise at least one chemotherapeutic compound, for example, for gene therapy, immunosuppressants, anti-inflammatory agents or immune enhancers, and for vaccines, chemotherapeutics including, but not limited to, gamma globulin, amantadine, guanidine, hydroxybenzimidazole, interferon-α, interferon-β, interferon-γ, tumor necrosis factor-alpha, thiosemicarbarzones, methisazone, rifampin, ribavirin, a pyrimidine analog, a purine analog, foscarnet, phosphonoacetic acid, acyclovir, dideoxynucleosides, a protease inhibitor, or ganciclovir.

The composition may also contain variable but small quantities of endotoxin-free formaldehyde, and preservatives, which have been found safe and not contributing to undesirable effects in the organism to which the composition is administered.

### Pharmaceutical Purposes

The administration of the composition (or the antisera that it elicits) may be for either a “prophylactic” or “therapeutic” purpose. When provided prophylactically, the compositions of the invention which are vaccines are provided before any symptom or clinical sign of a pathogen infection becomes manifest. The prophylactic administration of the composition serves to prevent or attenuate any subsequent infection.

When provided therapeutically, an attenuated or inactivated viral vaccine is provided upon the detection of a symptom or clinical sign of actual infection. The therapeutic administration of the compound(s) serves to attenuate any actual infection.

Thus, a vaccine composition of the present invention may be provided either before the onset of infection (so as to prevent or attenuate an anticipated infection) or after the initiation of an actual infection.

A composition is said to be “pharmacologically acceptable” if its administration can be tolerated by a recipient mammal. Such an agent is said to be administered in a “therapeutically effective amount” if the amount administered is physiologically significant. A composition of the present invention is physiologically significant if its presence results in a detectable change in the physiology of a recipient patient, e.g., enhances at least one primary or secondary humoral or cellular immune response against at least one pathogen, e.g., strain of an infectious influenza virus.

The “protection” provided need not be absolute, i.e., the influenza infection need not be totally prevented or eradicated, if there is a statistically significant improvement compared with a control population or set of mammals. Protection may be limited to mitigating the severity or rapidity of onset of symptoms or clinical signs of the pathogen, e.g., influenza virus infection.

### Pharmaceutical Administration

A composition of the present invention may confer resistance to one or more pathogens, e.g., one or more influenza virus strains, by either passive immunization or active immunization. In active immunization, a live vaccine composition is administered prophylactically to a host (e.g., a mammal), and the host's immune response to the administration protects against infection and/or disease. For passive immunization, the elicited antisera can be recovered and administered to a recipient suspected of having an infection caused by at least one pathogen.

In one embodiment, the vaccine is provided to a mammalian female (at or prior to pregnancy or parturition), under conditions of time and amount sufficient to cause the production of an immune response which serves to protect both the female and the developing embryo or newborn (via passive incorporation of the antibodies).

The present invention thus includes methods for preventing or attenuating a disease, e.g., an infection by at least one strain of pathogen. As used herein, a vaccine is said to prevent or attenuate a disease if its administration results either in the total or partial attenuation (i.e., suppression) of a clinical sign or condition of the disease, or in the total or partial immunity of the individual to the disease.

At least virus isolate of the present invention, which may also include a virus which is inactivated or attenuated, one or more isolated viral proteins thereof, one or more isolated nucleic acid molecules encoding one or more viral proteins thereof, or a combination thereof, may be administered by any means that achieve the intended purposes.

For example, administration of such a composition may be by various parenteral routes such as subcutaneous, intravenous, intradermal, intramuscular, intraperitoneal, intranasal, oral or transdermal routes. Parenteral administration can be accomplished by bolus injection or by gradual perfusion over time.

For instance, a typical regimen for preventing, suppressing, or treating an influenza virus related pathology, comprises administration of an effective amount of a vaccine composition as described herein, administered as a single treatment, or repeated as enhancing or booster dosages, over a period up to and including between one week and about 24 months, or any range or value therein.

According to the present invention, an “effective amount” of a composition is one that is sufficient to achieve a desired effect. It is understood that the effective dosage may be dependent upon the species, age, sex, health, and weight of the recipient, kind of concurrent treatment, if any, frequency of treatment, and the nature of the effect wanted. The ranges of effective doses provided below are not intended to limit the invention and represent dose ranges.

The dosage of a live virus vaccine for an animal such as a mammalian adult organism can be from about 10²-10¹⁵, e.g., 10³-10¹², plaque forming units (PFU)/kg, or any range or value therein. However, the dosage should be a safe and effective amount as determined by conventional methods, using existing vaccines as a starting point.

For raccoon pox viruses that express influenza virus HA, the dosage of PFU or immunoreactive HA in each dose of replicated virus vaccine may be standardized to contain a suitable amount, e.g., 30 to 100 μg or any range or value therein, or the amount recommended by government agencies or recognized professional organizations. If the raccoon pox virus expresses a different influenza virus protein, that protein may be standardized. For example, the quantity of NA may also be standardized, however, this glycoprotein may be labile during purification and storage.

The invention will be further described by the following non-limiting examples.

### Example 1

### Materials and Methods

### Viruses and Cells

Raccoonpox virus (RCN) was kindly provided by Dr. Joseph Esposito (Centers for Disease Control, Atlanta, Ga. RNA and formalin-inactivated viral proteins from the influenza A/Vietnam/1203/2004 (H5N1) virus were provided by Dr. Stacey Schultz-Cherry (Dept. of Medical Microbiology and Immunology, UW-Madison). Vero cells (Cercopithecus aethiops kidney, ATCC catalog #CCL-81) were used for propagation and titration of RCN. BS-C-1 cells (Cercopithecus aethiops kidney, ATCC catalog #CCL-26) were used for generation of recombinant RCN viruses followed by thymidine kinase knockout selection on Rat-2 cells (Rat embryo, ATCC # CRL-1764). Cells were cultured in Dulbecco's Modified Eagle's Medium (Invitrogen, Carlsbad, Calif.) with 5-10% fetal bovine serum (Gemini Bio-Products, West Sacramento, Calif.) and 1% antibiotic-antimycotic (HyClone, Logan, Utah). Primary chick embryo fibroblasts (CEF) were prepared from Gallus gallus eggs at 10 to 12 days of incubation (Sunnyside Hatchery, Beaver Dam, Wis.) and propagated in DMEM+10% FBS+1% antibiotic-antimycotic; primary CEF were tested mycoplasma-free by PCR before use in assays.

### Construction of Recombinant RCN-Ha and RCN-Luciferase Viruses

Total RNA was isolated from MDCK cells infected with HPAIV A/Vietnam/1203/2004. The HA gene was cloned with the primers HA-for: 5′-GCAGATTTGCATTGGTTACCATGC-3′ (SEQ ID NO:1) and HA-rev: 5′-TTAAATGCAAATTCTGCATTGTAACGATCC-3′ (SEQ ID NO:2). The forward primer was designed to truncate the 16-amino acid signal sequence of native HA for replacement with the human tissue plasminogen activator (tPA) signal sequence to increase secretion of the recombinant HA. The 20 μL reaction mix contained 0.5 μg total RNA, 4 pmol HA-rev primer, and 0.5 mM dNTPs and was incubated for 5 minutes at 65° C. then placed on ice. Then 4 μL 5× Superscript II RT PCR buffer, 2 μL 0.1 M DTT, and 1 μL RNaseOUT (Invitrogen) were added to the mixture and incubated for 2 minutes at 25° C. followed by addition of 1 μL Superscript II reverse transcriptase (Invitrogen). Reverse transcription was allowed to proceed for 50 minutes at 42° C. and was stopped by incubation at 70° C. for 15 minutes.

PCR amplification of the reverse transcription product was initiated by addition of 2.5 μL of the above reaction to a 50 μL mixture containing 1 μL each of 25 μM HA-for and HA-rev primers, 1.5 μL H₂O and 45 μL Platinum PCR Supermix (Invitrogen). Amplification was carried out by incubation at 94° C. for 2 minutes followed by 35 cycles of 94° C. for 2 minutes, 55° C. for 30 seconds and 72° C. for 3 minutes. The amplification product was cloned into plasmid pCR-4 TOPO vector as described in the TOPO TA cloning kit (Invitrogen, Carlsbad, Calif.). Colonies were screened for HA inserts by colony blotting and selected clones were sequenced and compared to the published HA sequence. In vitro mutagenesis using the QuikChange II site-directed mutagenesis kit (Stratagene, Cedar Creek, Tex.) was carried out to repair two discrepancies in the sequence between the cloned fragment and the published sequence. The HA gene was then excised from the pCR-4 vector by digestion with BamH I and Nhe I restriction enzymes and ligated into the pTK-IRES-tPA transfer plasmid in frame with the tPA coding sequence, to generate pTK-HA2.

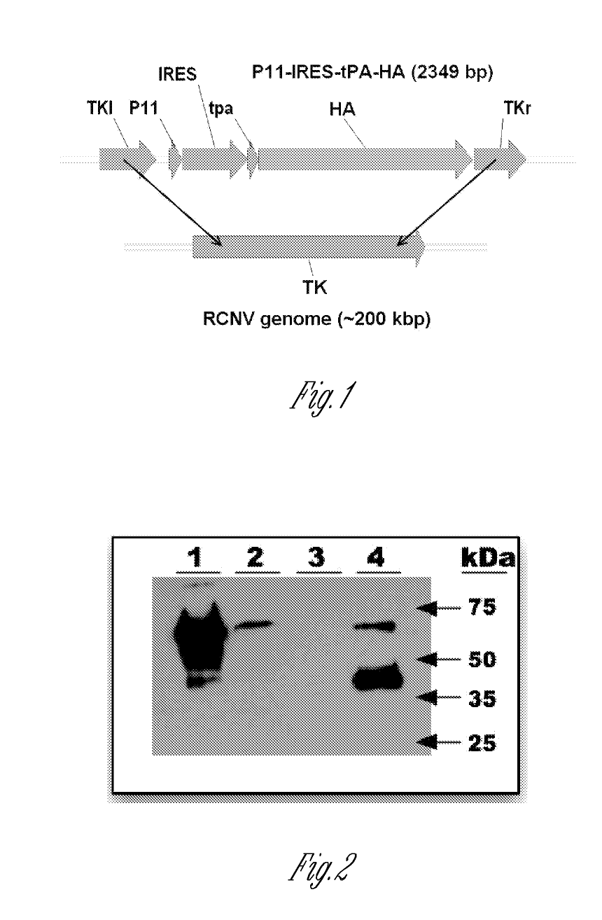

Monolayers of BS-C-1 cells were infected with RCN wild-type (wt) followed by transfection with pTK-HA2 in Lipofectamine transfection reagent (Invitrogen) to generate RCN-HA (FIG. 1). Virus clones with disruption of the tk gene by insertion of HA were selected during three rounds of growth on Rat-2 cells in media containing 50 μg/mL bromoedeoxyuridine (MP Biomedicals, Solon, Ohio). The presence of the HA gene sequence in RCN-HA was detected by PCR amplification of HA sequences using the original HA-for and HA-rev primers. Vero cells were infected with positive clones and cell lysates were analyzed by Western blotting to confirm expression of the HA construct.

Viral stocks for animal studies were prepared by growing one positive clone of RCN-HA on monolayers of Vero cells. Recombinant virus was harvested from cells after 72 hours by freeze-thawing cells three times, sonicating on ice for 3×15 seconds at 18 W (Sonicator 3000, Misonix, Farmingdale, N.Y.), followed by centrifugation on 36% sucrose cushions at 35,000×g for 80 minutes. Stocks were stored at −80° and titrated on Vero cells in 96-well microtiter plates prior to animal studies.

A recombinant RCN expressing firefly luciferase (luc) as a reporter gene was constructed and purified by similar methods. The luciferase gene was amplified by PCR from the pGLP3 plasmid (Promega, Madison, Wis.) and cloned into the pTK transfer plasmid. Similar procedures were followed as described above for the cloning and selection of the luc gene into RCN, with the exception that the vaccinia synthetic early-late promoter (SEL) was used to drive the expression of the luc gene and the tPA secretory signal was not included in the coding sequence.

### Western Blotting

Vero cell monolayers were infected with RCN-HA or RCN-TK⁻ at a multiplicity of infection (m.o.i). of 1, and harvested at 24 h.p.i. 25 μL aliquots were boiled in Laemmli buffer-BME, and separated on a 4% stacking, 10% resolving polyacrylamide gel. Proteins were transferred by electrophoresis to a nitrocellulose membrane overnight, stained with Ponceau to detect protein-standards, and probed with anti-HA mouse monoclonal antibody (BEI Resources, Manassas, Va.). For chemiluminescent detection, the anti-mouse-IgG HRP-conjugated secondary antibody (KPL) was used, followed by luminol substrate (Thermo Fisher, Waltham, Mass.).

CEF viral-infected samples (see below) were first concentrated by methanol precipitation from a 150 μL volume before boiling, electrophoresis, and transfer as described above. The nitrocellulose membrane was probed with pooled antiserum from mice vaccinated with recombinant H5 HA protein. For colorimetric detection, an anti-mouse-IgG alkaline phosphatase-conjugated secondary antibody (KPL, Gaithersburg, Md.) was used, followed by BCIP/NBT substrate (Thermo Fisher).

### Replication Assay

Replication assays of RCN-HA in Vero and CEF cells were carried out by using a modified version of the method described for fowlpox virus by Weli et al. (2005). Briefly, 3×10⁶ cells were seeded in 10 cm polystyrene tissue culture plates (BD Biosciences, San Jose, Calif.) and allowed to adhere overnight. For these studies, m.o.i.s of 0.01 and 0.1 were used to infect Vero and CEF cells, respectively. At 2 hours p.i., monolayers were washed twice with PBS, overlaid with 8 mL DMEM+2% FBS, and incubated at 37° C. Cells and supernatants were harvested at 2, 24, 48, 72, and 120 h p.i. Supernatants were aspirated and stored separately. Then, monolayers were scraped and resuspended into 2 mL DMEM. Both supernatant and cell suspension samples were sonicated 3×15 seconds with 45 second intervals at 18 W on ice and titrated on Vero cells. 150 μL aliquots of the CEF-grown samples were also analyzed by Western blot as described above.

### Mice

In a preliminary mouse study, the efficacy of the RCN-HA vaccine was tested by the intramuscular (IM) and intranasal (IN) routes in 4-week old NJ mice (Jackson Laboratories, Bar Harbor, Me.) (Table 2). In addition, groups of mice were also vaccinated with purified, recombinant hemagglutinin (BEI Resources) as a positive control, and with empty viral vector (RCN-TK) as a negative control. Serum samples were collected from each mouse by saphenous vein bleed at day 14, and 24 days after vaccination. All of the mice received a booster of the same vaccination, dose and route at 41 days after initial vaccination, and were challenged 67 days after initial vaccination with 10 LD₅₀ of A/Vietnam/1203/04 (H5N1) highly pathogenic avian influenza.

TABLE 2
Vaccine doses and routes for evaluation of RCN-HA immunogenicity and
protection in mice.

| | | | | Vaccination Dose |
|---|---|---|---|---|
| Group | Number of animals | Treatment | Route | (prime/boost) |
| 1 | 5 | RCN/HA | IM | 10⁶ PFU |
| 2 | 2* | RCN/HA | IN | 10⁶ PFU |
| 3 | 5 | RCN-TK⁻ | IM | 10⁶ PFU |
| 4 | 4 | HA Protein | IM | 10 μg protein |

*Three mice died due to improper handling; unrelated to experimental treatment.
In the second mouse study, groups of ten 5 week old A/J mice (Jackson Laboratories, Bar Harbor, ME) were vaccinated with RCN-HA via the intramuscular (IM) or the intradermal (ID) route (Table3). Control groups received formalin inactivated H5N1 of highly pathogenic avian influenza virus (Vietnam 1203/2004) or the empty vector (RCN/TK knockout) as positive and negative controls, respectively. All animals received a booster vaccination at day 42 post-primary vaccination. Immunized animals were then challenged 4 weeks post-booster vaccination via the intranasal route with 10 LD₅₀s of Vietnam 1203 H5N1 highly pathogenic avian influenza.

TABLE 3
Vaccine doses and routes for evaluation of RCN-HA immunogenicity and
protection in mice.

| Group | Treatment | Route | Vaccination Dose |
|---|---|---|---|
| 1 | Formalin Inactivated | IM | Prime/Boost (5 ug) |
| | H5N1 avian influenza | | |
| | virus (positive control) | | |
| 2 | RCN/HA (prime & boost) | ID | Prime/Boost |
| | | | (5 × 10\hat{ }6 PFU) |
| 3 | RCN/HA (prime & boost) | IM | Prime/Boost |
| | | | (1 × 10\hat{ }7 PFU) |
| 4 | RCN/HA | ID | Prime only (5 × 10\hat{ }6 PFU) |
| 5 | RCN/HA | IM | Prime only (1 × 10\hat{ }7 PFU) |
| 6 | RCN TK knockout | IM | Prime/Boost |
| | (negative control) | | (1 × 10\hat{ }7 PFU) |

### Safety of Raccoon Pox Virus as a Potential Vector for Vaccines

Vaccinia virus, one of the widely used pox viruses in vaccination, has been reported to be unsafe in immunocompromised individuals, leading to search for safer second generation pox viral candidates for vaccine development. The safety of a raccoon pox viral vector was tested in SCID mice.

Groups of six SCID mice were infected intraperitoneally with the empty raccoon pox viral vector (RCN/TK knockout), wild-type raccoon pox virus (RCNwt) or with vaccinia (Wyeth strain). The animals were monitored for 12 weeks, weekly body weights were recorded and animals showing body conditioning score less than 2 (BCS<2) were euthanized.

### In Ovo Inoculation and Luciferase Assays

RCN-wt is known to form small, discrete, nonhemorrhagic pocks on the chorioallantoic membrane of chicken eggs (Alexander et al., 1972). To test the suitability of recombinant RCN as an in ovo vaccination vector, 10⁶ pfu of RCN-luc was injected either into the allantoic or yolk sacs of embryonated chicken eggs (Sunnyside Hatchery; n=8 per treatment); RCN-TK was used as a vector control (n=2). Embryos that died <24 hours post-infection were discarded as the deaths were assumed to be from mechanical injury from injection. At 24 hours and 48 h p.i., samples of allantoic fluid and embryo tissue were harvested and assayed for luciferase activity. From each embryo, samples of pectoral muscle, skin, and gut tissue were collected (approximately 0.5 g total). An equal volume (v/w) of sterile PBS was then added and the samples were freeze-thawed three times and homogenized with micro pestles (Argos Technologies, Elgin, Ill.). Homogenates were centrifuged briefly to pellet solids and the supernatants were used for virus titration and luciferase assays. RCN-HA in allantoic fluid and tissue homogenates was titrated on Vero cells. To measure luciferase activity, 100 μL of the suspension was further diluted 1:5 in cell culture lysis reagent (Promega, Madison, Wis.). 10 μL of allantoic fluid and tissue homogenate samples were used in a 96-well plate luciferase activity assay from a commercial kit (Luciferase Assay System, Promega).

### Biophotonic Imaging of RCN-Luc in Chickens

In vivo biophotonic imaging is a powerful tool for following the kinetics and distribution of recombinant vectors or pathogens in real time. However, this technique has not yet been reported for avian models. The use of luciferase to trace poxvirus distribution in mice has been reported for MVA-luc (Ramirez et al., 2003); in vivo bioluminescence of monkeypox-luc was also correlated with viral titers in two rodent species (Osorio et al., 2009) and RCN-luc has also been tested in mice. To examine the tissue tropism of RCN, groups of three 7-day-old SPF chicks (Hy-Vac AdeI, IA), were inoculated with 10⁶ pfu RCN-luc in 100 μL by intramuscular (IM) injection in the right thigh or intradermal (ID) injection in the right wing-web. Vector-control chicks (n=2) received 10⁶ PFU of RCN-HA, and negative controls (n=4) received 100 μL PBS. On days 1, 3, 5, and 7 post-infection, all chicks were weighed to monitor growth. RCN-injected chicks were imaged in the IVIS 200 imager (Caliper Life Sciences, Alameda, Calif.). Chicks were anesthetized with 1.5-2% isoflurane in 2 L/minute oxygen and injected with luciferin in the wing vein, 10 μL/g of body mass at a concentration of 150 mg/kg luciferin (Caliper Life Sciences) dissolved in PBS. Immediately after injection, chicks were placed in the imager with their right sides facing upward and imaged over 2 to 8 minutes with an f/stop of 2. Living Image software (Caliper Life Sciences) was used to draw equal-sized regions of interest (ROI) over the injection sites and to normalize raw data (counts) to radiance in units of photons/s/cm²/steradian.

### Chicken Vaccination

Groups of eleven, 2-week-old White Leghorn chickens (Gallus gallus, Sunnyside Hatchery Inc., Beaver Dam, Wis.) were vaccinated either IM or orally (PO) with 4×10⁷ PFU of RCN-HA; or by oculonasal instillation (ON) with 2×10⁷ PFU; or by wing-web stabber (WW) with 4×10⁶ PFU (Table 4). Three control groups received IM injections of phosphate-buffered saline (PBS, 100 μL-negative control); RCN-TK (5×10⁶ PFU, vector control); and formalin-inactivated HPAIV A/Vietnam/1203/04 (H5N1) with 1% Alhydrogel® adjuvant (13.6 μg in 100 uL, positive control). At four weeks post-initial (PI) vaccination, all groups received booster vaccinations (same dose and route). Blood samples were collected at 2, 3, 4, 5, 6, and 8 weeks of age for determination of anti-RCN and anti-influenza HA serum titers by ELISA. Chickens were not challenged with HPAIV for biosafety reasons.

TABLE 4
Vaccine doses and routes for evaluation of RCN-HA immunogenicity
in chickens (n = 11 for all groups).

| | | | Dose |
|---|---|---|---|
| Group | Vaccine | Volume and route | (prime and boost) |
| 1 | Phosphate-buffered | 100 μL IM | n/a |
| | saline control | | |
| 2 | RCN-TK⁻ | 100 μL IM | 5 × 10⁶ PFU |
| 3 | Formalin-inactivated | 100 μL IM | 13.6 μg |
| | A/Vn/1203/04 | | |
| 4 | RCN-HA | 100 μL IM | 4 × 10⁷ PFU |
| 5 | RCN-HA | 10 μL ID (wing-web) | 4 × 10⁶ PFU |
| 6 | RCN-HA | 100 μL oral | 4 × 10⁷ PFU |
| 7 | RCN-HA | 100 μL oculonasal | 2 × 10⁷ PFU |

### Serology

Serum samples were obtained by allowing blood to clot at 4° C. followed by centrifugation at 10,000×g for 10 minutes. For biosafety reasons, microneutralization assays were not performed with A/Vn/1203/04 (H5N1); instead, an anti-HA ELISA was performed to determine IgY serum titers. A titration of the coating antigen (recombinant H5 HA cloned from A/Vn/1203/04, BEI Resources) was first performed to optimize the amount used in the assays. 96-well EIA plates (Corning Life Sciences, Lowell, Mass.) were coated with 4 ng/well of HA in 100 μL phosphate-buffered saline, pH 7.4 (PBS), overnight at 4° C. Plates were washed three times with 200 μL/well wash buffer (PBS containing 0.1% Tween 20) and blocked for 1 hour at room temperature (22-24° C.) with 100 μL of blocking buffer (PBS containing 0.1% Tween 20, 2% FBS, and 5% skim milk). Serial dilutions (1:160 to 1:10240) of serum samples made in blocking buffer were applied to the plates in 100 μL/well volumes in duplicate. Normal chicken serum (Sigma-Aldrich) diluted to 1:160 was used as a negative control. After a 1 hour incubation at room temperature, plates were washed again. The secondary antibody (HRP-conjugated rabbit anti-chicken IgY, Bethyl Laboratories, Montgomery, Tex.) was diluted in blocking buffer and applied to the plates. Following a 1 hour incubation and washing, 100 μL/well of TMB peroxidase substrate (KPL) was added to the plate. After 5-10 min, the reaction was stopped by addition of 100 μL/well of 0.5 N H₂SO₄ Plates were read on an ELx800 microplate reader (BioTek, Winooski, Vt.) at 450 nm. The signal intensity of duplicate sample dilutions was averaged by the BioTek Gen5 software. Dilutions with an OD of 3 standard deviations above the average of the negative controls were considered positive.

To measure anti-RCN IgY titers, EIA plates were coated with 5×10⁴ PFU/well RCN-wt in carbonate coating buffer (15 mM Na₂CO₃, 35 mM NaHCO₃, pH 9.0) overnight at 4° C. and fixed with 10% formalin in PBS. The assay was carried out using the same protocol as the anti-HA ELISA.

A commercial test kit (FlockChek* Avian Influenza Antibody Test Kit, IDEXX Laboratories, Westbrook, Me.) was used to determine if RCN-HA vaccinated chickens could be distinguished from inactivated influenza-vaccinated chickens. The contents of the kit are proprietary but the coating antigen is described as “solubilized inactivated native virus” [personal communication from IDEXX technical support].

### Statistical Analysis

GraphPad Prism 5 (La Jolla, Calif.) was used to perform statistical analyses and generate graphs of data. ELISA IgG/IgY titers were log-transformed before comparison using an unpaired t-test with Welch's correction. For biophotonic imaging data, Living Image (Caliper Life Sciences) was used to quantify photon emission from live chick tissue.

### Results

In the studies described herein, a recombinant raccoon pox virus was used that encoded the hemagglutinin (HA) gene (RCN-HA) from a recent, highly pathogenic avian influenza (HPAI) isolate, expressed from a promoter and with secretory signals previously shown to increase expression of antigen. The immunogenicity and efficacy of RCN-HA for protection against a lethal HPAI challenge was evaluated in a mouse model.

The suitability of RCN as a vaccine vector for chickens was also assessed. To do this, influenza hemagglutinin was used as a model antigen as well as luciferase as a reporter gene. The replication and gene expression of RCN-HA was first characterized in vitro in cell culture. In addition, RCN-luc was used both in chicken eggs to examine expression in ovo and for biophotonic imaging in live chicks. Finally, the safety and immunogenicity of RCN-HA was tested by several routes of administration in young chicks.

### Expression of HA Protein by RCN-HA in Mammalian Cells

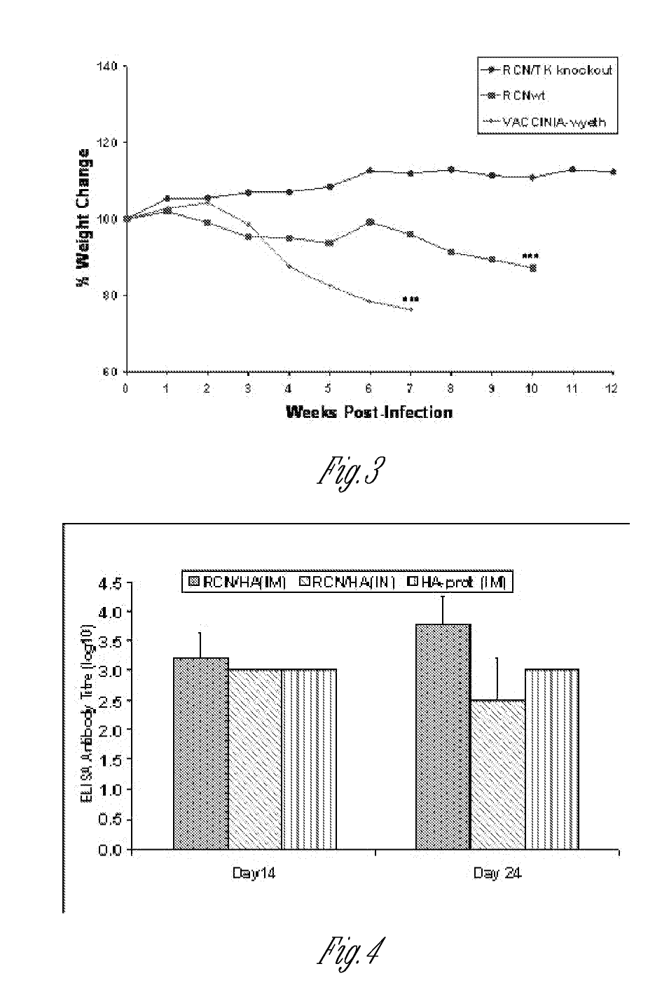

At 24 hours post infection, Western blot analysis showed that RCN-HA expresses high levels of HA protein in vitro (FIG. 2). 25 μl of unconcentrated cellular lysate from monolayers infected at a low m.o.i. produced a large quantity of HA compared to the 0.2 μg of purified HA protein in lane 4. A sucrose gradient-purified RCN-HA preparation contained a small quantity of HA protein, less than 0.2 μg in 2.5×10⁷ PFU of virus (FIG. 2, lane 3).

### Safety of Raccoon Pox Virus as a Potential Vector for Vaccines

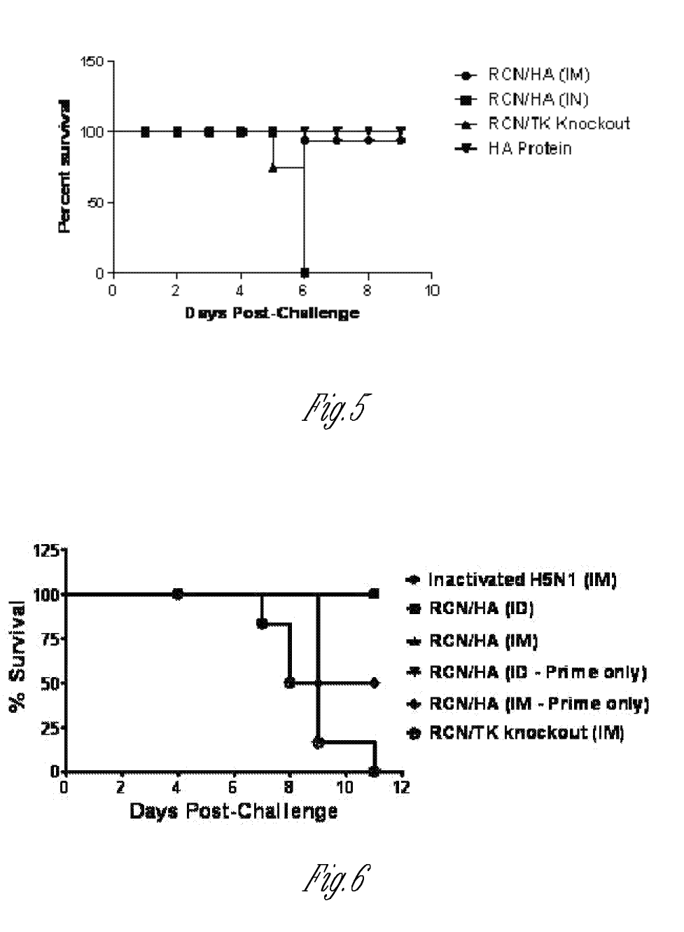

All SCID mice in the vaccinia-Wyeth and the wild type RCN groups developed pox lesions on their tails and feet and showed persistent weight loss, disease and death. Only one animal from the RCN viral vector group developed lesions and died. Weight losses in the vaccinia-Wyeth and the wild type RCN groups were significantly greater (P<0.0001) than the group that was infected with the RCN viral vector, however, there was no significant difference (P>0.05) between the wild type RCN virus and the vaccinia-Wyeth groups (FIG. 3).

Except for a “take” (small vaccination site lesion) in the wing-web vaccinated group, no dermal or mucosal lesions were observed in chicks vaccinated with RCN-HA at two and six weeks of age. At eight weeks of age, no significant differences in body mass between RCN-HA vaccinated groups, inactivated influenza vaccinated group and PBS control group were observed.

### Immunogenicity of RCN-HA in Mice

The anti-HA immune response induced by the RCN-HA and recombinant HA protein indicates that the RCN-based vaccine is highly immunogenic (FIG. 4). At 24 days PI, the RCN-HA vaccine delivered IM induced significantly higher antibody titers (3.8 log₁₀) than the recombinant HA-IM delivered vaccine (3.2 log₁₀). The RCN-HA vaccine was also highly immunogenic by the IN route.

Protection from Lethal Challenge in Mice

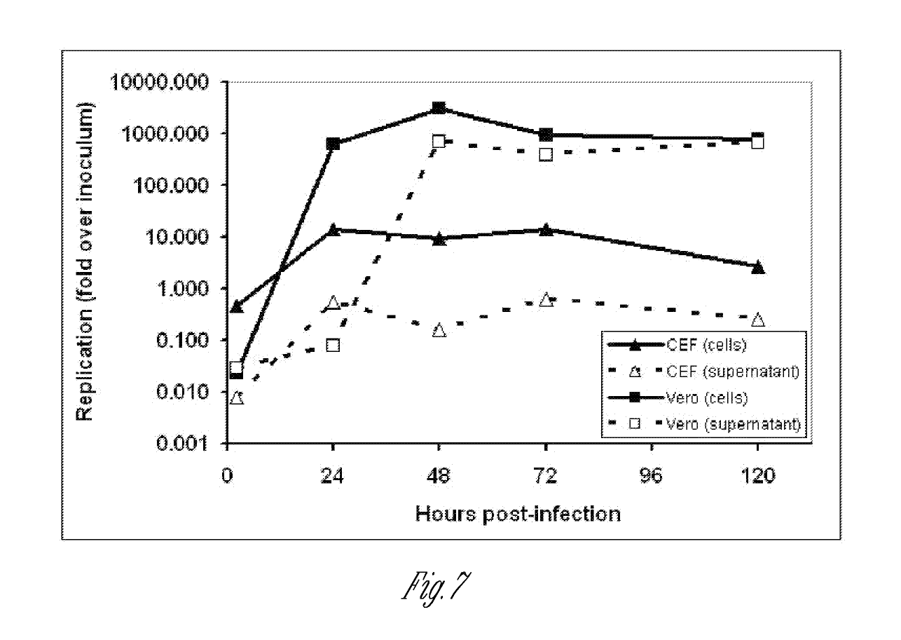

In the first study, 4/5 mice vaccinated with RCN-HA by the IM route survived a lethal challenge with HPAI, compared to 5/5 mice vaccinated with recombinant HA protein (FIG. 5). Vaccine administered by the IN route was not protective (2/2 mice died). Immunized mice were challenged via the IN route by inoculating mice with 1×10⁴ (10 LD₅₀). Differences in survival rates between challenged groups were analyzed by Kaplan-Meier method with the log-rank (Mantel-Cox) test.

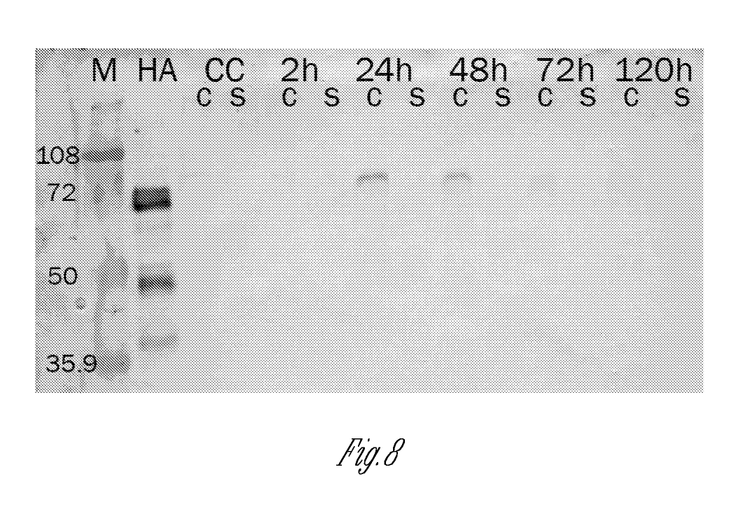

The second study was more comprehensive. Animals that received a single dose of the RCN-HA construct via the IM route had 50% survival rate after challenge with a median survival time of 10 days, however, mice that received prime and booster vaccinations of the RCN-HA construct via the IM route had 100% survival rate (FIG. 6). Protection offered by the single dose of RCN-HA via the IM route was not significantly lower (P>0.05) than protection offered by the double dose treatment. Vaccination via the ID route offered 100% protection to animals that received either single or double doses of the RCN-HA construct. There was a 100% survival rate for mice immunized with the inactivated H5N1 while all the mice inoculated with the empty vector (RCN/TK knockout) were not protected. Protection offered by the RCN-HA construct or the inactivated H5N1 virus was significantly higher (P<0.01) than animals that received the empty vector (RCN/TK knockout), the negative control.

Thus, when administered to BALB/c mice, RCN-HA was comparable to recombinant HA protein in terms of inducing anti-HA ELISA titers (data not shown). A single ID dose of RCN-HA or two IM doses protected 100% of mice from challenge with 10 mouse LD₅₀ of A/Vn/1203/04 HPAI. This level of protection was comparable to that conferred by two IM doses of inactivated A/Vn/1203/04.

### Growth of RCN-HA and Expression of HA Protein in Avian Cells

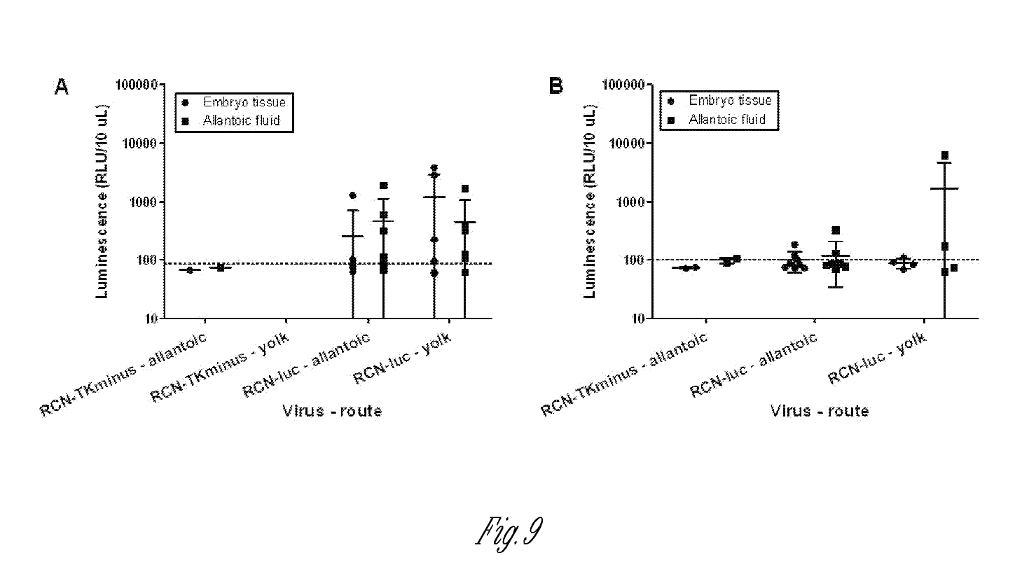

To determine if RCN can infect avian cells, the growth and expression of RCN-HA in CEFs was examined. RCN-HA replicates at very low levels in primary CEF compared to Vero cells (FIG. 7). In contrast to the large, lytic plaques seen in infected Vero monolayers within 24 to 48 hours postinfection, infected CEFs did not form lytic plaques and only became pycnotic and produced CPE at several days post-infection. Very low levels of RCN-HA virus were detected in the supernatant of infected CEFs. Expression of HA protein was detected at 24 h p.i. and persisted as long as 120 h p.i. (FIG. 8). No HA was found in the supernatant.

### Luciferase Expression in Ovo

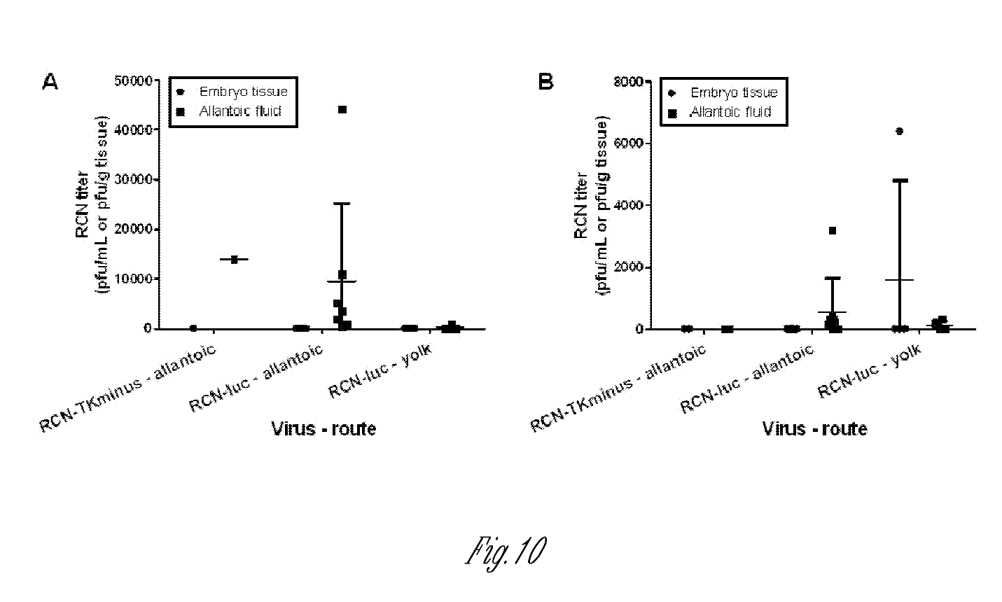

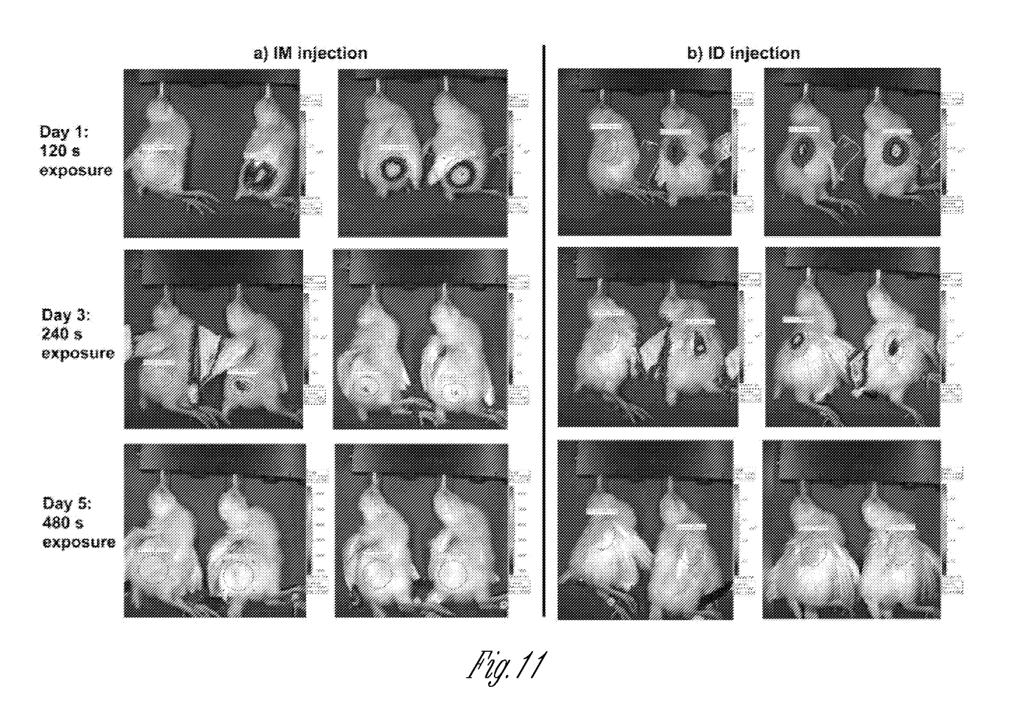

When measured at 24 hours post-inoculation, luciferase activity was detected in tissues and allantoic fluid in embryos that received 10⁶ pfu of RCN-luc into the allantoic and yolk sacs (FIG. 9A). However, at 48 hours post-infection luciferase expression had decreased to below detectable levels in most of the embryos inoculated by either route (FIG. 9B). RCN-luc in allantoic fluid was detected at higher levels in eggs inoculated by the yolk sac route than the allantoic sac route at 24 hours, and decreased at 48 hours (FIG. 10). No live virus was recovered from embryo tissues, except for one embryo inoculated by the yolk sac route. The low level of RCN-luc replication in CEF and embryonated eggs supports early reports indicating that wild-type RCN does not grow well in the chorioallantoic membrane (Alexander et al., 1972; Thomas et al., 1975). However, this low viral growth in CEFs did not impair expression of recombinant genes from a synthetic early-late (SEL) vaccinia promoter for recombinant RCN viruses.

### Biophotonic Imaging of RCN-Luc in Chickens

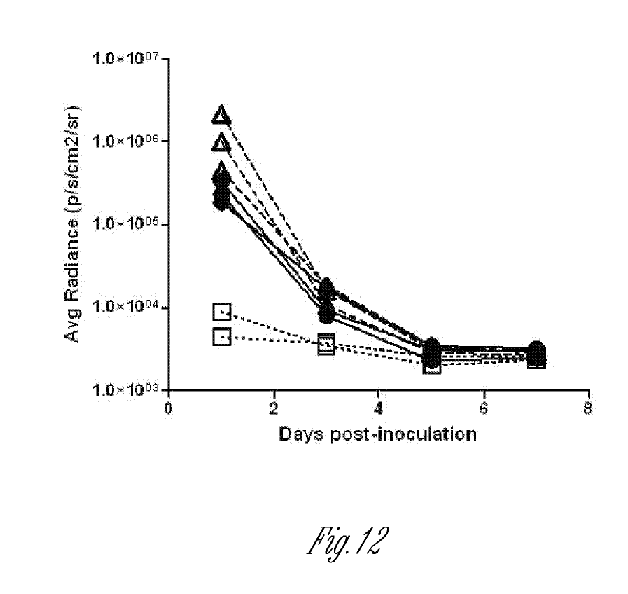

All three chicks inoculated with 10⁶ PFU of RCN-luc by the IM and ID routes developed a strong luminescent signal at 24 hours post-infection that persisted at least up to 3 d.p.i., but was undetectable by 5 d.p.i. (FIGS. 11-12). The luminescence was observed only at the site of inoculation and was more intense in birds inoculated by the ID route. No luminescence was seen in RCN-HA chicks (non-luminescent vector control). It is important to note that feathers did not appear to block the luminescent signal or display strong autoluminescence that could interfere with luc detection.

### Safety of RCN in Young Chickens

In the biophotonic imaging study, no lesions were observed in the RCN-luc IM group and a slight thickening of the wing-web injection site was observed in the ID group. The growth of chicks inoculated with RCN-luc was not impaired compared to an untreated control group, from 7 days to 21 days of age (data not shown). Except for a “take” (small vaccination site lesion) in the wing-web vaccinated group, no dermal or mucosal lesions were observed following prime and boost vaccinations of chicks with RCN-HA. At eight weeks of age, no significant differences in body mass were observed between the groups vaccinated with RCN-HA by the wing-web (843±64.3 g), oral (842±63.5 g), or oculonasal (780±77.7 g) routes and the inactivated influenza positive control group. (840±48.6 g). Chicks in the RCN-HA intramuscular group had a somewhat higher mean body mass (931±51 g, P=0.0027). The RCN-TK vector control group (860±78.9 g) and the PBS control group (887±51.8 g) were not significantly different from the inactivated influenza group.

### Immunogenicity of RCN-HA in Chickens

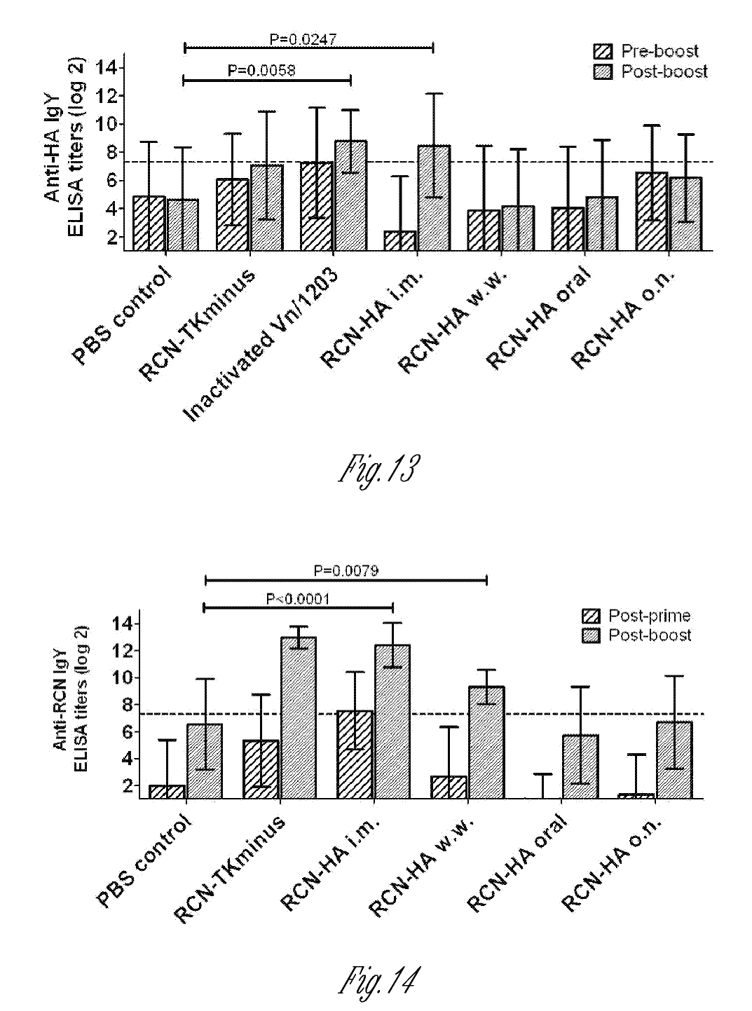

Humoral response to HA: The chicks used in this study were obtained from a conventional hatchery prior to being transferred to a secure facility at 1 day of age. While most of the sampled chicks were anti-HA seronegative prior to first vaccination at 2 weeks of age, low levels of IgY (1:160) were detected in 4 of 35 animals suggesting the presence of passively transferred maternal immunoglobulins. 2 weeks following booster vaccination, chickens vaccinated with RCN-HA by the IM route developed strong mean antibody titers that were comparable to those raised by the adjuvanted inactivated influenza vaccine and significantly higher (P=0.0247) than the PBS negative control group (FIG. 13). In contrast, the PO and oculonasal routes elicited poor levels of anti-HA antibodies that were not significantly different from the PBS control. No anti-HA responses were observed in the WW vaccinated group.

Humoral response to vector: RCN was highly immunogenic in chickens by the IM and WW (intradermal) routes and produced anti-RCN IgY titers of 1:640 to 1:40960 at 1 week post-prime vaccination and 1:640 to 1:2560 2 weeks after the booster vaccination (FIG. 14). It is worthwhile to mention that chicks vaccinated by the WW route received a tenfold lower dose compared to the IM group due to the volume limitation of the bifurcated needle vaccinator. Chicks vaccinated by oral or ON instillation did not develop anti-RCN IgY titers significantly different from the PBS control group.

Reaction with Commercial AI Test Kit

Serum samples from 2 weeks post-boost vaccination were tested with the FlockChek* Avian Influenza Antibody Test Kit (IDEXX) to determine if chicks vaccinated with RCN-HA could be differentiated from those vaccinated with inactivated influenza virus. 6 of 11 (55%) of chicks that received inactivated virus tested positive, whereas no samples from any of the RCN-HA groups or the two control groups tested positive. Therefore, vaccination with RCN-HA is compatible with the use of commonly used serological assays to detect birds that have actually been infected with avian influenza virus, whereas vaccination with an inactivated influenza vaccine causes a positive reaction.

### Discussion

Raccoon pox virus has previously been evaluated as a recombinant vaccine vector against rabies (Osorio et al., 2003b; Lodmell et al., 1991; DeMartini et al., 1999) and plague (Mencher et al., 2004; Rocke et al., 2005) in various mammalian species.

Although RCN expressing rabies and plague antigens was highly immunogenic by mucosal routes in cats and prairie dogs, respectively, this was not the case for RCN-HA in chickens. Nevertheless, a strong immune response was observed when RCN was administered by IM and ID routes, as well as excellent protection from HPAI challenge in mice after only one dose by the ID route. Further optimization of HA expression (e.g., by changing the promoter and/or secretory signal) may improve immunogenicity, and the addition of other influenza antigens (e.g., neuraminidase (NA) and/or nucleoprotein (NP)) may enhance protection by targeting other parts of the viral life cycle and by lowering the dose required to protect against challenge.

Domestic cats and other felines are highly susceptible to avian influenza (Keawcharoen et al., 2004; Thiry et al., 2007). The immunogenicity of recombinant RCN by the IN route in cats shows that RCN-HA may be a suitable vaccine for rapid, needle-free delivery in companion animals, and wild or captive felines that are valuable from a conservation standpoint. Needle-free delivery to a mucosal surface and immunogenicity with a single dose is a clear advantage of RCN over fowlpox as a vaccine vector for felines (Karaca et al., 2008).

These studies also demonstrated that recombinant RCN viruses can infect avian cells and successfully express foreign antigens in embryos as well as live chicks. Using a recombinant RCN containing the HA gene of highly pathogenic avian influenza virus (H5N 1) strong expression was detected in both Vero cells and primary CEFs. In contrast to high viral titers obtained following RCN-HA infection of mammalian (Vero) cells, one step-growth studies in CEFs, showed low viral yields, suggesting limited replication of RCN in avian cells. Additional evidence of viral growth and expression in chickens was obtained using biophotonic in vivo imaging and a recombinant RCN containing the luc gene. Following either IM or ID administration of RCN-luc, strong luciferase expression was detected for at least 3 days post-infection, showing that RCN can infect chickens and achieve high levels of antigen expression. When administered to chicks, RCN-HA elicited humoral anti-HA IgY antibody responses comparable to those observed by alum-adjuvanted inactivated H5N1 influenza virus.

Of particular interest is the ability of RCN to express the HA gene of HPAI viruses. HPAIV is a pathogen of great concern in veterinary medicine and has the potential to cause a human pandemic with high mortality (see Cumulative Number of Confirmed Human Cases of Avian Influenza A/(H5N1) Reported to WHO; 2009 (Apr. 2, 2009)) and concurrent global economic impacts. Unfortunately, generation of an inactivated vaccine from an autogenous AI isolate (i.e., an isolate from the same outbreak) can take weeks to months (Hehme et al., 2008; Hehme et al., 2002). Production of inactivated influenza vaccines is also limited by the supply of SPF chicken eggs as a substrate for growing virus, which may be very low in the event of an HPAI pandemic (Murakami et al., 2008). There are also biosafety concerns since H5N1 HPAI viruses must be produced under BSL-3 conditions and are a hazard to workers. Conventional vaccines are also difficult to stockpile and transport because formalin-inactivated vaccines can lose activity when stored at room temperature (Fatunmbi et al., 1993). Finally, the use of inactivated whole-virus formulations can interfere with DIVA serosurveillance since vaccinates will have antibodies against the matrix (M) and nucleoprotein (NP) proteins, which are detected by commercial AI test kits (Capua et al., 2007).

Viral vectors such as RCN can avoid some of the problems associated with conventionally-produced influenza vaccines. Following recombinant viral infection of target cells, newly expressed antigens are processed and presented by professional antigen-presenting cells, resulting in the induction of antibody responses with high avidity, as well as major histocompatibility complex class I-restricted cytotoxic T-lymphocyte (CTL). Because of this well-balanced induction of B and T cell responses, they do not require the use of adjuvants to induce strong immune responses. In addition, RCN can enable field investigations that require distinguishing between vaccinated and naturally infected animals using common assays such as agar gel precipitin or ELISA (Bublot et al., 2006). RCN could have further advantages compared to recombinants based on attenuated strains of avian pathogens such as Newcastle Disease virus (NDV) and fowlpox virus (FPV) because of concerns regarding pre-existing immunity to the vector (Swayne et al., 2000). Since Newcastle Disease and fowlpox are common diseases in domestic poultry, natural infection or vaccination may limit the use of recombinant NDV-HA and FPV-HA to young, naïve birds that have not been immunized, exposed, or have maternal antibodies to NDV and FPV. In addition, some poultry species such as domestic ducks are resistant to fowlpox and require 100 chicken dose-equivalents to stimulate a strong immune response (Steensels et al., 2007).

The present data also showed that recombinant RCN is safe for use in young chicks since no spread of luminescence away from the inoculation sites was detected and the growth of chicks was not impaired compared to controls. The potential for using recombinant RCN for in ovo vaccination was tested by injection of eggs with RCN-luc. Strong luciferase activity was observed in samples of allantoic and amniotic fluid as well as embryo tissues. The use of luciferase to trace poxvirus infection has been validated for the recombinant vaccinia strain NYVAC-luc and MVA-luc (Ramirez et al., 2003; Gomez et al., 2007) and monkeypox-luc (Osorio et al., 2009). This is the first demonstration of non-invasive real-time biophotonic imaging in chicks in vivo (as opposed to fluorescent microscopy of avian embryos which are not a good model for infectious disease research) Since young chicks at 8 days of age and weighing ≦70 g were able to tolerate anesthesia and luciferin injection well, bioluminescent imaging may be a valuable tool for other avian models such as Japanese quail.

While the majority of existing AI vaccines are parenterally administered, mucosal immunization, e.g. instillation into the eye, nares, or oral cavity, is desirable because secretory IgA antibodies can block infection at the site of entry (Pan et al., 2009) and provide better cross-protection against heterologous influenza viruses than does IgG (Ichinohe et al., 2008; Bright et al., 2008). In addition, mucosal vaccination can be more rapidly administered for emergency prophylaxis in an AI outbreak. Inactivated vaccine is not immunogenic by this route unless coadministered with an adjuvant as a TLR agonist (Ichinohe et al., 2008). In this study, RCN-HA did not stimulate serum IgY responses by mucosal routes of administration. Saliva and lacrimal fluid samples were not obtained, so it is unknown whether this vaccine induced secretory IgA responses. It is also possible that higher viral doses than those used in the present study are required to induce stronger immune responses by mucosal routes in chickens. Mucosal immunization against HPAI has been demonstrated with a recombinant human adenoviral vaccine (van Ginkel et al., 2009). Ad5-HA was shown to induce high anti-H5 hemagglutination inhibition (HI) titers in serum when administered to chickens by intranasal or ocular routes, and B cells secreting IgG and IgA specific to both H5 and adenovirus were found in the Harderian glands.

These studies also demonstrate that RCN can infect and express reporter genes in chicken embryos. In ovo vaccination of chick embryos in the poultry industry offers significant advantages, including rapid onset of protection from diseases that can occur soon after hatching, less trauma to chicks, and labor-saving (Johnston et al., 1997). This method is already used for several common poultry diseases (e.g. Marek's Disease (Sharma et al., 1982), Newcastle Disease) Dilayeris et al., 2007), infectious bursal disease (Haygreen et al., 2006), and coccidiosis (Lillehoj et al., 2005)). Embryos inoculated with the Ad5-HA vaccine developed high HI titers post-hatch and were protected from morbidity and mortality upon H5N2 HPAI challenge (Toro et al., 2009). Therefore, in ovo injection may be an efficient method to protect commercially raised poultry against HPAI.

Compared to other viral vectored vaccines for HPAI in poultry, RCN-HA has several favorable characteristics. It can be propagated to high titers in Vero cells, which are an FDA-approved substrate for vaccine production. This circumvents the problem of SPF egg supply that exists for the conventional flu vaccine and for MVA and fowlpox recombinants, and does not require a proprietary helper cell line as does the replication-deficient Ad5-HA. Moreover, the use of raccoon pox rather than fowlpox as a vector for delivering influenza HA may also overcome the problem of preexisting immunity to vector in the existing commercial fowlpox-AI vaccine (Swayne et al., 2000).

In summary, recombinant RCN is capable of expressing genes of interest in birds in vivo and stimulating an immune response, and is safe in young chicks. RCN has potential as a new viral vector for use in poultry vaccination, which is compatible with AI surveillance and should not be affected by preexisting immunity against common poultry pathogens.

The large genome size of RCN, its safety in various species and its growth characteristics in tissue culture make it an efficient vector for production of recombinant live viral vaccines. RCN-HA is also compatible with DIVA surveillance strategies, since uninfected vaccinated animals would not be seropositive for other pathogen proteins, e.g., other influenza viral proteins, such as nucleoprotein or matrix (Swayne et al., 2007).

### Example II

To determine whether expression of additional antigens in RCN vectors can provide enhanced protection, reduce the dose needed for protection and/or provide cross (heterologous) protection, RCN-NP and RCN-NA constructs were prepared. NP is highly conserved throughout HPAI strains, and would potentially offer a wider range of protection. Moreover, the use of a combination of antigens is also more likely to induce both cellular and humoral immune responses and thus will increase protection compared to current inactivated vaccine technologies that only stimulate humoral responses.

RCN based influenza vaccines expressing influenza neuraminidase (RCN-NA), and nucleoprotein (RCN-NP) from A/Vn/1203/04(H5N1) were tested for protein expression. Mice are then immunized with RCN-HA, RCN-NP and RCN-NA and the humoral (antibody) immune response is determined by ELISA or microneutralization assays of mouse serum. The cellular immune response is detected by ELISPOT assays of mouse splenocytes. Several weeks after vaccination, mice are challenged with a lethal dose of either the homologous (A/Vn/1203/04, clade 1) or heterologous (A/Indonesia/5/05) viruses. Mouse survival, health status, and influenza virus lung titers are determined.

For example, groups of ten, 4-6 week old AJ mice are vaccinated intradermally with single or combined RCN-flu constructs at a dose of 5×10⁷ pfu in 50 μL (Table 4). Mice are bled at 2 and 4 weeks post-initial (PI) vaccination. At 4 weeks PI, mice are challenged with 1×10⁴ TCID₅₀ units (10 MLD₅₀) of either A/Vietnam/1203/04 (clade 1, experiment #1) or A/Indonesia/5/05 (clade 2, experiment #2). At day 4 post-challenge, 3 mice from each group are sacrificed, and blood and lung tissues are harvested. All remaining mice are sacrificed at day 14 post-challenge and tissues harvested as on day 4 PI. Microneutralization tests are performed on all serum samples to assess neutralizing antibody production. Neuraminidase assays utilizing MUNANA substrate are performed to determine NA specific antibody production. Viral titers and histopathology are analyzed from lung tissues.

TABLE 4
Experimental design to test the efficacy of RCN-flu constructs in mice.

| | | Number | | | |
|---|---|---|---|---|---|
| Experiment | | of | | | |
| # | Group | Animals | Vaccination | Route | Challenge Virus |
| 1 | 1 | 10 | RCN/HA | ID | A/Vietnam/ |
| | 2 | 10 | RCN/HA, | ID | 1203/04 |
| | | | RCN/NP | | |
| | 3 | 10 | RCN/HA, | ID | |
| | | | RCN/NA | | |
| | 4 | 10 | RCN/HA, | ID | |
| | | | RCN/NP, | | |
| | | | RCN/NA | | |
| | | | Combo | | |
| | 5 | 10 | Inactivated | IM | |
| | | | H5N1 | | |
| | | | Primary | | |
| | 6 | 10 | RCN-TK− | ID | |
| 2 | 7 | 10 | RCN/HA | ID | (A/Indonesia/ |
| | 8 | 10 | RCN/HA, | ID | 5/05) |
| | | | RCN/NP | | |
| | 9 | 10 | RCN/HA, | ID | |
| | | | RCN/NA | | |
| | 10 | 10 | RCN/HA, | ID | |
| | | | RCN/NP, | | |
| | | | RCN/NA | | |
| | | | Combo | | |
| | 11 | 10 | Inactivated | IM | |
| | | | H5N1 | | |
| | | | Primary | | |
| | 12 | 10 | RCN-TK− | ID | |

### Example III

To determine if expression of an adjuvant can provide enhanced protection, reduce the dose needed for protection and/or provide cross (heterologous) protection, the adjuvant effect of RCN-LTB which expresses heat-labile cholera enterotoxin subunit B (LTB), a Toll-like receptor (TLR) stimulator, on RCN-HA administration is assessed. LTB has been shown to increase levels of proinflammatory cytokines when administered in vivo by acting on TLRs. RCN-LTB is employed as a recombinant adjuvant since it has been shown to enhance the mucosal response against flu antigens. A mixed formulation at varying doses is administered to mice to determine which provides the best protection. In addition, mice are bled at weekly intervals to characterize the kinetics of the immune response. Several weeks after vaccination, mice are challenged with a lethal dose of either the homologous (A/Vn/1203/04, clade 1) or heterologous (A/Indonesia/5/05) viruses. Mouse survival, health status, and influenza virus lung titers are then determined.

For instance, doses of RCN-HA (optionally with NA and/or NP) varied from 5×10⁵ to 5×10⁷ PFU are administered with, or without RCN-LTB. Groups of 10, 4-6 week old AJ mice are vaccinated intradermally as shown in Table 5. Mice are bled, challenged and samples analyzed as discussed above.

TABLE 5
Experimental design to test the efficacy of
RCN-flu/RCN-LTB constructs in mice

| Exper- | | Number | | | | |
|---|---|---|---|---|---|---|
| iment | | of | | Vaccine | | Challenge |
| # | Group | Animals | Vaccination | Dose | Route | virus |
| 1 | 1 | 10 | RCN/Flu- | 5 × 10⁷ | ID | A/Vietnam/ |
| | | | best | | | 1203/04 |
| | 2 | 10 | RCN/Flu- | 5 × 10⁷, | ID | |
| | | | best, | 5 × 10⁷ | | |
| | | | RCN/LTB | | | |
| | 3 | 10 | RCN/Flu- | 5 × 10⁶ | ID | |
| | | | best | | | |
| | 4 | 10 | RCN/Flu- | 5 × 10⁶, | ID | |
| | | | best, | 5 × 10⁷ | | |
| | | | RCN/LTB | | | |
| | 5 | 10 | RCN/Flu- | 5 × 10⁵ | ID | |
| | | | best | | | |
| | 6 | 10 | RCN/Flu- | 5 × 10⁵, | ID | |
| | | | best, | 5 × 10⁷ | | |
| | | | RCN/LTB | | | |
| | 7 | 10 | Inactivated | | IM | |
| | | | H5N1 | | | |
| 2 | 8 | 10 | RCN/Flu- | 5 × 10⁷ | ID | (A/Indonesia/ |
| | | | best | | | 5/05) |
| | 9 | 10 | RCN/Flu- | 5 × 10⁷, | ID | |
| | | | best, | 5 × 10⁷ | | |
| | | | RCN/LTB | | | |
| | 10 | 10 | RCN/Flu- | 5 × 10⁶ | ID | |
| | | | best | | | |
| | 11 | 10 | RCN/Flu- | 5 × 10⁶, | ID | |
| | | | best, | 5 × 10⁷ | | |
| | | | RCN/LTB | | | |
| | 12 | 10 | RCN/Flu- | 5 × 10⁵ | IM | |
| | | | best | | | |
| | 13 | 10 | RCN/Flu- | 5 × 10⁵, | | |
| | | | best, | 5 × 10⁷ | | |
| | | | RCN/LTB | | | |
| | 14 | 10 | Inactivated | | ID | |
| | | | H5N1 | | | |

### Example IV

Since RCN-HA conferred protection against a lethal HPAI challenge in mice, and was immunogenic in chickens, the efficacy of RCN-influenza constructs is assessed in chickens. The immunogenicity and efficacy of RCN-flu constructs in poultry are tested on specific pathogen-free (SPF) White Leghorn chicks (Hy-Vac, AdeI, IA). Groups of 12 one-week-old chicks receive vaccinations as outlined in Table 6. At 4 week post-prime vaccination, all chicks receive a booster vaccination of the same vaccines(s) and dose previously administered. Blood samples are obtained from the jugular vein on day 0 (prior to vaccination), and at 2, 4, and 6 week post-initial vaccination. Chicks are weighed at weekly intervals to monitor growth.

At 6 week post-prime, 4 chicks per group are euthanized to collect spleens and Harderian glands for cytokine (qRT-PCR) and immunoglobulin (IgY and IgA ELISPOT) assays. The remaining groups of 8 chicks are transferred to an ABSL-3 facility (USGS National Wildlife Health Center, Madison, Wis.) and challenged with 10⁵ EID₅₀ of either A/Vn/1203/04 or A/Indonesia/5/05 by instillation into the choana. Body mass, condition, and mortality are monitored for 2 weeks following challenge. Oropharyngeal and cloacal swabs to determine shedding of AIV are obtained at 2, 4, and 7 day post-challenge. At the end of the 2 week period any surviving chicks are euthanized.

TABLE 6
Evaluation of RCN vaccines in chickens.

| Experiment | | | | |
|---|---|---|---|---|
| # | Group | Vaccine | Route | Challenge |
| 1 | 1 | RCN- | IM | A/Vietnam/1203/ |
| | | TK−(negative | | 2004 |
| | | control) | | |
| | 2 | Inactivated | IM | |
| | | A/Vietnam/ | | |
| | | 1203/2004 | | |
| | | influenza | | |
| | | virus (positive | | |
| | | control) 10 ug | | |
| | 3 | RCN-flu | IM | |
| | | combination | | |
| | 4 | RCN-flu | ID (wing- | |
| | | combination | web) | |
| | 5 | RCN-flu | IN | |
| | | combination | (intranasal) | |
| 2 | 6 | RCN- | IM | (A/Indonesia/ |
| | | TK−(negative | | 5/05) |
| | | control) | | |
| | 7 | Inactivated | IM | |
| | | A/Vietnam/ | | |
| | | 1203/2004 | | |
| | | influenza | | |
| | | virus (positive | | |
| | | control) 10 ug | | |
| | 8 | RCN-flu | IM | |
| | | combination | | |
| | 9 | RCN-flu | ID (wing- | |
| | | combination | web) | |
| | 10 | RCN-flu | IN | |
| | | combination | (intranasal) | |

Given the strong IgY response to RCN-HA, chicks vaccinated by the IM and ID routes will likely have high anti-HA titers comparable to chicks vaccinated with inactivated influenza. Chicks vaccinated by the IN route will likely have high anti-HA IgA titers in tears but may not have IgA or IgY antibodies in serum. Based on previous results (above), vaccination by the IM and ID routes will likely provide 100% protection from lethal HPAI challenge and the IN route may provide significantly better protection than the negative control. RCN-flu constructs administered by all three routes likely induce strong cellular immune responses in the spleen, while administration via the IN route likely induces the strongest cellular response in the Harderian gland.

### REFERENCES

Alexander et al., J. Wildl. Dis., 8:119 (1972).
Anonymous. Pandemics and pandemic threats since 1900. Department of Health and Human Services (www.pandemicflu.gov); 2006.
Binns and Smith, ed., Recombinant Poxviruses, CRC Press, 1992.
Blanton et al., Vaccine, 25:7296 (2007).
Bright et al., PLoS ONE, 3:e1501 (2008).
Bright et al., Vaccine, 25:3871 (2007).
Bublot et al., Ann. N.Y. Acad. Sci., 1081:193 (2006).
Capua and Cattoli, Dev. Biol. (Basel), 130:137 (2007).
Capuaa and Alexander, Vaccine, 26:D70 (2008).
Carallaro et al., Virology, 190:434 (1992).
Chan et al., Virology, 380:304 (2008).
Cottingham et al., PLoS ONE, 3:e1638 (2008).
Cumulative Number of Confirmed Human Cases of Avian Influenza A/(H5N1) Reported to WHO. 2009; 2009(2 Apr. 2009).
De et al., Vaccine, 6:257 (1988).
de Jong et al., Nature, 389:554 (1997).
DeMartini et al., Arch. Virol., 133:211 (1993).
Dilayeris et al., Vaccine, 25:3792 (2007).
Domi et al., Proc. Natl. Acad. Sci. USA, 99:12415 (2002).
Dothager et al., Curr. Opin. Biotechnol., 20:45 (2009).
E I Garch et al., Vet. Immunol. Immunopathol., 123:230 (2008).
Elroy-Stein and Moss, Curr. Protoc. Protein Sci., 5:5.15 (2001).
Falkner and Moss, J. Virol., 64:3108 (1990).
Fatunmbi et al., Avian Dis., 37:639 (1993).
Fauci, A. S., Emerq. Infect. Dis., 12:73 (2006).
Fekadu et al., J. Wildl. Dis., 27:681 (1991).
Fenner, F., In: Binns M M, Smith G L, ed., Recombinant Poxviruses, Boca Raton, Fla., CRC Press, 1:1 (1992).
Gilbert et al., Vaccine, 24:4554 (2006).
Gomez et al., J. Gen. Virol., 88:2473 (2007).
H5N1 avian influenza: Timeline of major events. 2009.
Haygreen et al., Vaccine, 24:4951 (2006).
Hehme et al., Med. Microbiol. Immunol., 191:203 (2002).
Hehme et al., Vaccine, 26:D23 (2008).
Hu et al., Vaccine, 1997; 15:1466 (1997).
Ichinohe et al., Expert Rev. Vaccines, 7:1435 (2008).
Johnston et al., Poult. Sci., 76:165 (1997).
Karaca et al., Clin. Diaqn. Lab. Immunol., 12:1340 (2005).
Keawcharoen et al., Emerq. Infect. Dis., 10:2189 (2004).
Krauss et al., J. Gen. Viral., 83:2347 (2002).
Laddy, PLoS ONE, 3:e2517 (2008).
Larson and Schultz, Vet. Ther., 8:101 (2007).
Lillehoj et al., Avian Dis., 49:112 (2005).
Lodmell et al., J. Virol., 65:3400 (1991).
Lüschow et al., Vaccine, 19:4249 (2001).
Lutze-Wallace et al., Virus Genes, 10:84 (1995).
Mackett et al., Proc. Natl. Acad. Sci. USA, 79:7415 (1982).
McLeod, A., Economic and social impacts of avian influenza.
Meltzer et al., Emerq. Infect. Dis., 5:659 (1999).
Mencher et al., Infect. Immun., 72:5502 (2004).
Moss, B., In: Binns M M, Smith G L, ed., Recombinant Poxviruses, Boca Raton, Fla., CRC Press, 45:45 (1992).
Murakami et al., J. Virol., 82:10502 (2008).
Nolan et al., Vaccine, 26:4160 (2008).
Omar et al., Vet. Immunol. Immunopathol., 62:73 (1998).
Osorio et al., J. Drug Target., 11:463 (2003b).
Osorio et al., Vaccine, 21:1232 (2003a).
Osorio et al., Plos One, 4(8):e6592 (2009).
Pan et al., Vaccine, 27:1018 (2009).
Paoletti, E., Proc. Natl. Acad. Sci. USA, 93:11349 (1996).
Ramirez et al., Arch. Virol., 2003; 148(5):827-39.
Rocke et al., J. Wildl. Dis., 44:930 (2008).
Ropp et al., J. Clin. Microbiol., 33:2069 (1995).
Sharma and Burmester, Avian Dis., 26:134 (1982).
Shi et al., Vaccine 25:7379 (2007).
Skinner et al., Expert Rev. Vaccines, 4:63 (2005).
Spatz et al., J. Gen. Virol., 75:1235 (1994).
Spread of Avian Flu Could Affect Next Year's Economic Outlook. East Asia Update—Countering Global Shocks 2005.
Steensels et al., Avian Dis., 51:325 (2007).
Suarez and Schultz-Cherry, Dev. Comp. Immunol., 24:269 (2000).
Suarez et al., Dev. Biol. (Basel), 124:117 (2006).
Swayne et al., Avian Dis., 44:132 (2000).
Swayne et al., Avian Dis., 51:697 (2007).
Swayne, Comp. Immunol. Microbiol. Infect. Dis., epub 2008 Apr. 26.
Swayne, D. E., Ann. N.Y. Acad. Sci., 1081:174 (2006).
Taubenberger et al., Nature, 437:889 (2005).
Taylor et al., Vaccine, 6:504 (1988).
Taylor et al., Virology, 187:321 (1992).
Tewalt et al., PLoS Pathoq., 5:e1000457 (2009).
Thiry et al., Vet. Microbiol., 122:25 (2007).
Thomas et al., Arch. Virol., 49:217 (1975).
Tizard, I., Seminars in Avian and Exotic Pet Medicine, 11:2 (2002).
Toro et al., Poult. Sci., 88:867 (2009).
Tumpey et al., J. Clin. Microbiol., 43:676 (2005).
van Ginkel et al., Dev. Comp. Immunol., 33:28 (2009).
Veits et al., Proc. Natl. Acad. Sci. USA, 103:8197 (2006).
Veits et al., Vaccine, 26:1688 (2008).
Wasmoen et al., Adv. Exp. Med. Biol., 380:221 (1995).
Weli et al., Virus Res., 109:39 (2005).
World Organisation for Animal Health, In: Anonymous Manual of Diagnostic Tests and Vaccines for Terrestrial Animals 2008, p. 465 (2008).
Wu et al., Clin. Infect. Dis., 48:1087 (2009).

All publications, patents and patent applications are incorporated herein by reference. While in the foregoing specification, this invention has been described in relation to certain embodiments thereof, and many details have been set forth for purposes of illustration, it will be apparent to those skilled in the art that the invention is susceptible to additional embodiments and that certain of the details herein may be varied considerably without departing from the basic principles of the invention.

1. A vaccine comprising a live recombinant raccoon pox virus, the genome of which comprises at least one expression cassette having a promoter operably linked to a heterologous open reading frame comprising a nucleotide sequence for an influenza virus antigen that induces the production of neutralizing antibodies to the antigen in an animal administered an effective amount of the recombinant virus.

2. The vaccine of claim 1 further comprising an adjuvant.

3. The vaccine of claim 1 further comprising a different recombinant virus.

4. The vaccine of claim 3 wherein the different recombinant virus is a different live recombinant raccoon pox virus.

5. The vaccine of claim 1 which is adapted for mucosal or in ovo delivery.

6. The vaccine of claim 1 wherein the heterologous open reading frame encodes a fusion protein comprising the influenza virus antigen.

7. The vaccine of claim 6 wherein the fusion protein comprises a heterologous secretory peptide.

8. The vaccine of claim 6 wherein the fusion comprises a heterologous membrane anchoring peptide.

9. The vaccine of claim 1 wherein the influenza antigen comprises influenza HA, NA, NP or M.

10. The vaccine of claim 9 wherein the HA is H5.

11. A composition comprising two or more live recombinant raccoon pox viruses, wherein the genome of a first live recombinant raccoon pox virus comprises a first expression cassette having a promoter operably linked to a heterologous open reading frame comprising a nucleotide sequence for an antigen of a pathogen, and wherein the genome of a second live recombinant raccoon pox virus comprises a second expression cassette having a promoter operably linked to a heterologous open reading frame comprising a nucleotide sequence for a protein.

12. The composition of claim 11 wherein the second expression cassette encodes an adjuvant.

13. The composition of claim 11 wherein the second expression cassette encodes a different antigen of the pathogen.

14. The composition of claim 11 wherein the second expression cassette encodes an antigen of a different pathogen.

15. A method to immunize an animal against pathogen infection or replication, comprising: administering to a nonmammalian animal or an egg thereof, a composition comprising an amount of at least one live recombinant raccoon pox virus, the genome of which comprises at least one expression cassette having a promoter operably linked to a heterologous open reading frame comprising a nucleotide sequence for an antigen of the pathogen effective to induce an adaptive immune response to the pathogen.

16. A method to immunize an animal or an egg thereof against influenza virus infection or replication, comprising: administering to an animal or an egg thereof a composition comprising an amount of at least one live recombinant raccoon pox virus, the genome of which comprises at least one expression cassette having a promoter operably linked to a heterologous open reading frame comprising a nucleotide sequence for an influenza virus antigen, effective to induce an adaptive immune response to influenza virus.

17. The method of claim 15 or 16 wherein the animal is an avian.

18-20. (canceled)

21. The method of any of claim 15 or 16 wherein the recombinant virus is intradermally, intramuscularly or mucosally administered.

22-31. (canceled)
